(19) United States
(12) Patent Application Publication
(10) Pub. No.: US 2026/0354948 A1
(43) Pub. Date: Nov. 12, 2026

(54) ADJUSTING A PRELIMINARY THREE-DIMENSIONAL DIGITAL JAW MODEL

(71) Applicant: EXOCAD GMBH, Darmstadt (DE)

(72) Inventor: Tillmann STEINBRECHER, Seeheim-Jugenheim (DE)

(21) Appl. No.: 19/202,600

(22) Filed: May 8, 2025

(51) Int. Cl. A61C 13/00; G06T 19/20

(52) U.S. Cl. CPC A61C 13/0004; G06T 19/20; G06T 2210/41; G06T 2219/2008

(57) ABSTRACT

Disclosed is a method for adjusting a preliminary three-dimensional digital jaw model of at least a part of a jaw of a patient. The method comprises receiving scan data of a jaw with implants arranged therein, which was acquired using a scanning method. The preliminary three-dimensional digital jaw model of the at least part of the jaw is received, which is descriptive of preliminary positions of reference elements of the jaw. Measuring data of an additional measurement of relative positions of the reference elements of the jaw are received. The preliminary three-dimensional digital jaw model is deformed. The deforming results in an adjusted three-dimensional digital jaw model with adjusted positions of the reference elements using the measured relative positions of the reference elements described by the measuring data as references for the deforming.

### FIELD OF THE INVENTION

The invention relates to the field of dental technology. More particularly, the invention relates to a method, a computer program product, and a computer device for adjusting a preliminary three-dimensional digital jaw model of at least a part of a jaw of a patient.

### BACKGROUND

Modern CAD/CAM approaches for generating dental prostheses use three-dimensional digital models of the intraoral tissue, at which the respective dental prostheses are to be arranged. However, acquiring a precise three-dimensional digital model of intraoral tissue may be challenging.

### SUMMARY OF THE INVENTION

The objectives underlying the invention are solved by the features of the independent claims.

In one aspect, a method for adjusting a preliminary three-dimensional digital jaw model of at least a part of a jaw of a patient is disclosed. The method comprises receiving the preliminary three-dimensional digital jaw model of the at least part of the jaw. The preliminary three-dimensional digital jaw model is descriptive of preliminary positions of reference elements of the jaw. Measuring data of an additional measurement of relative positions of the reference elements of the jaw are received. The preliminary three-dimensional digital jaw model is deformed. The deforming results in an adjusted three-dimensional digital jaw model with adjusted positions of the reference elements using the measured relative positions of the reference elements described by the measuring data as references for the deforming.

Examples may enable a provision of a precise three-dimensional digital jaw model of at least a part of a jaw. Deviations of the preliminary three-dimensional digital jaw model from the form of the actual jaw, e.g., resulting from scan data and/or a processing of scan data used for generating the preliminary three-dimensional digital jaw model, may be corrected by the deforming. Such deviations due to a processing of the scan data may, e.g., result from an assembling of patches of imaging data of a surface of the at least part of the jaw. In particular, deviations of the preliminary positions of the reference elements, e.g., preliminary implant positions of reference elements in form of implants, from the actual positions of the reference elements in the at least part of the jaw may be corrected. The adjusted positions of the reference elements may precisely coincide with the actual positions of the reference elements. Thus, an adjusted three-dimensional digital jaw model resulting from the deforming may be provided, which precisely represents a geometric form of the surface of the at least part of the jaw. In particular, the adjusted three-dimensional digital jaw model may precisely represent the positions of the reference elements, e.g., implants arranged in the at least part of the jaw.

Such an adjusted three-dimensional digital jaw model may enable a generation of a three-dimensional digital prosthesis model of an implant-based dental prosthesis, which fits accurately onto the surface of the at least part of the jaw. Furthermore, the three-dimensional digital prosthesis model may be provided with receptions for receiving fastening means configured for fastening the implant-based dental prosthesis to implants, e.g., implants used as reference elements for adjusting the preliminary three-dimensional digital jaw model. Such receptions may, e.g., be accurately arranged within the three-dimensional digital prosthesis model, when the precise positions of the implants within the at least part of the jaw are identified by the three-dimensional digital jaw model.

A generating of the preliminary three-dimensional digital jaw model may, e.g., comprise an assembling the imaging data of a plurality of patches of a surface of the at least part of the jaw. The assembling of the patches of the surface of the at least part of the jaw may comprise errors to some degree, which may add up. In case a first patch is provided, and a second patch is registered with respect to the first patch, a first error may occur. The first error may be descriptive of an error of position of a second surface section described by the second patch relative to an adjacent first surface section described by the first patch. When the second patch with the first error is used as a reference for registering a third patch with respect to this second patch, a second error may occur. However, since the position of the second patch already comprises an error, an error of position of a third surface section described by the third patch relative to the first surface section described by the first patch, i.e., a total error of position of the third surface section, may be a sum of the first and second error. Consequently, errors may, e.g., increase from patch to patch. In particular, at a distal end of the preliminary three-dimensional digital jaw model errors may add up. In order to correct this effect, the preliminary three-dimensional digital jaw model may be deformed using the relative positions of the reference elements as references for the deforming. This may have the effect that not only the surface of the at least part of the jaw may be resembled more accurately than without the deformation. In addition, the positions of the reference elements, e.g., implants, within the at least part of the jaw may be identified particularly precisely by the adjusted positions of the adjusted, i.e., deformed three-dimensional digital jaw model.

The preliminary three-dimensional digital jaw model may, e.g., be generated using scan data of the at least part of the jaw. Examples may allow to use a scanning method for acquiring the scan data, which requires an assembling of scanned patches, like an optical intraoral scanning method, and at the same time to achieve a high global accuracy. A scanning method based on an assembling of scanned patches may achieve a higher local accuracy, i.e., a higher accuracy regarding the structures resembled by the individual patches. However, the assembling may result in inaccuracies regarding the arrangement of the highly accurate patches relative to each other. This may reduce a global accuracy. With the deforming of the preliminary three-dimensional digital jaw model using a measuring method configured for precisely determining relative positions of reference elements within the at least part of the jaw, a three-dimensional digital jaw model may be provided which combines the benefits of both methods, i.e., scanning method and measuring method, with each other. This may result in an adjusted three-dimensional digital jaw model, which is locally as well as globally highly accurate.

Examples may enable an adjusting of a preliminary three-dimensional digital jaw model using measuring data acquired with a measuring method, which is better suited for a precise determining of relative positions of reference elements, like implants in a jaw bow, than a scanning method used for providing scan data for generating the respective jaw model. In particular, e.g., only scan and measuring data of the at least part of the jaw may be used for the generating and/or for the adjusting, i.e., deforming, of the preliminary three-dimensional digital jaw model. For example, no other models, like any three-dimensional digital prosthesis models, may be used for this purpose.

Examples may avoid issues arising from small inaccuracies in an image registration process of patches of scan data which may lead to significant deviations in the preliminary three-dimensional digital jaw model. In particular, problems resulting from such deviations when creating a dental prosthesis, like an implant-based dental bridge, may be avoided. It may, e.g., be avoided that inaccuracies in the three-dimensional digital jaw model cause the dental prosthesis to not fit properly onto a patient's jaw bow.

Examples may refer to a computer-implemented method for generating and/or adjusting a three-dimensional digital jaw model, i.e., a preliminary three-dimensional digital jaw model of at least part of a patient’s jaw bow, in particular an edentulous part of the jaw bow, with reference elements, like implants. The method may start with executing a scanning method and acquiring scan data of a surface of the at least part of the jaw. The scan data acquired using the scanning method may, e.g., be used for generating the preliminary three-dimensional digital jaw model. Alternatively, the preliminary three-dimensional digital jaw model may be generated in any other way. Subsequently, relative positions of the implants within the at least part of the jaw may be determined independently of the scan data. For example, measuring data may be acquired using a measuring method. The measuring method is configured for capturing the relative positions of the reference elements of the jaw, which are then utilized as references for an adjustment of the preliminary three-dimensional digital jaw model. The adjusting comprises a deforming of the preliminary three-dimensional digital jaw model. The preliminary digital jaw model is deformed using the relative positions as references to ensure an accurate alignment of the positions of the reference elements in the three-dimensional digital jaw model with their actual positions in the patient's jaw. The resulting adjusted three-dimensional digital jaw model may serve as a foundation for further procedures, such as a generating of a three-dimensional digital prosthesis model, e.g., of an implant-based dental bridge, which may be configured to be arranged onto the reference elements, e.g., implants.

For example, the at least part of the jaw of the patient is at least a part of a jaw bow of the patient. For example, the preliminary three-dimensional digital jaw model may cover only a subsection of the jaw bow. For example, the preliminary three-dimensional digital jaw model may cover a single hemisphere of the jaw bow, i.e., half the jaw bow. For example, the preliminary three-dimensional digital jaw model may cover less than half the jaw bow. For example, the preliminary three-dimensional digital jaw model may cover more than half the jaw bow. For example, the adjusted three-dimensional digital jaw model may cover only a subsection of the jaw bow. For example, the adjusted three-dimensional digital jaw model may cover a single hemisphere of the jaw bow, i.e., half the jaw bow. For example, the adjusted three-dimensional digital jaw model may cover less than half the jaw bow. For example, the adjusted three-dimensional digital jaw model may cover more than half the jaw bow.

For example, a plurality of preliminary three-dimensional digital jaw models may be provided, which in combination may cover the full jaw bow. For example, the preliminary three-dimensional digital jaw models of the plurality of preliminary three-dimensional digital jaw models may be individually adjusted. The adjusting of the preliminary three-dimensional digital jaw models may result in a plurality of individually adjusted three-dimensional digital jaw models, which may cover the full jaw bow. For example, the individually adjusted three-dimensional digital jaw models of the plurality of individually adjusted three-dimensional digital jaw models may be combined to provide a single combined adjusted three-dimensional digital jaw model of the full jaw. For example, the measurement data used for individually adjusting the preliminary three-dimensional digital jaw models of the plurality of preliminary three-dimensional digital jaw models may be measurement data covering all the reference elements comprised by the jaw bow. For example, the same measurement data may be used for individually adjusting all the preliminary three-dimensional digital jaw models of the plurality of preliminary three-dimensional digital jaw models. For example, individual measurement data may be provided for individually adjusting the preliminary three-dimensional digital jaw models of the plurality of preliminary three-dimensional digital jaw models. The individual measurement data may, e.g., be measurement data covering only the reference elements comprised by a subsection of the jaw bow which is covered by the individual preliminary three-dimensional digital jaw model to be adjusted using the respective individual measurement data.

For example, the preliminary three-dimensional digital jaw model may be a preliminary three-dimensional digital jaw model of a jaw bow, i.e., a full jaw bow of the patient. For example, the preliminary three-dimensional digital jaw model of the jaw bow may be generated using scan data of the jaw bow. For example, the adjusted three-dimensional digital jaw model may be an adjusted three-dimensional digital jaw model of a jaw bow, i.e., a full jaw bow of the patient.

For example, the at least part of the jaw may be an edentulous part of the jaw. For example, the at least part of the jaw may be at least a part of an edentulous jaw bow. For example, the at least part of the jaw may comprise one or more teeth and/or tooth parts, like tooth stamps. Examples may enable a provision of accurate three-dimensional digital jaw models of at least partially edentulous jaw bows as well as of jaw bows comprising one or more teeth and/or tooth parts.

The term jaw bow refers to intraoral tissue within an oral cavity of a patient comprised by a bow-shaped jaw, e.g., maxilla and/or mandible, of the patient. The intraoral tissue may comprise soft and/or hard intraoral tissue of the patient's jaw. The term may relate to the curved alignment or arch of the jaw tissues, encompassing both soft and/or hard jaw tissue. Hard jaw tissue may, e.g., comprise underlying bony structures, such as the maxilla (upper jaw) and mandible (lower jaw), which form the anatomical framework of the oral cavity. Furthermore, in case of a non-edentulous jaw, the hard jaw tissue may, e.g., comprise one or more teeth and/or teeth parts comprised by the respective jaw. Soft jaw tissue may, e.g., comprise mucosal lining, gingiva, also referred to as gums, and/or other intraoral tissues that covers and/or interacts with the bony structures. Such a jaw bow may be understood as the bow-like shape the jaw formed by the alveolar ridge, i.e., the bony ridge containing the tooth sockets, and surrounding soft jaw tissue, like the gingiva. It may, e.g., be significant in dental assessments, treatment planning, and a generating of prosthetics or orthodontic appliances to ensure functionality, comfort, and structural harmony in the patient's oral cavity.

For example, the preliminary three-dimensional digital jaw model may be provided by a three-dimensional digital mesh structure. The mesh structure may, e.g., be a polygon mesh. For example, the preliminary three-dimensional digital jaw model may be provided by a three-dimensional digital point cloud. The point cloud may comprise a discrete set of data points in space which represent a three-dimensional shape, e.g., a three-dimensional shape of at least a part of the jaw of the patient.

The reference elements may, e.g., comprise elements of hard intraoral tissue of the patient's jaw, like natural teeth, tooth sections, and/or tooth stumps. The reference elements may, e.g., comprise artificial elements arranged within the patient's jaw, like implants and/or elements arranged on implants, like abutments and/or dedicated physical fiducial markers. The artificial elements arranged within the patient's jaw may, e.g., comprise dental prostheses or dental prosthesis elements arranged within the patient's jaw. The dental prostheses or dental prosthesis elements arranged within the patient's jaw may, e.g., be implant based or arranged on natural hard intraoral tissue of the patient's jaw. The artificial elements arranged within the patient's jaw may, e.g., comprise dedicated markers arranged within the patient's jaw for the purpose of the scan data and/or measuring data acquisition. The reference elements may, e.g., comprise elements of soft intraoral tissue of the patient's jaw, like gingiva section. The reference elements may, e.g., comprise dedicated markers arranged on soft intraoral tissue of the patient's jaw for the purpose of the scan data and/or measuring data acquisition.

For example, the deforming of the preliminary three-dimensional digital jaw model is executed iteratively. The deforming may, e.g., be executed iteratively until the adjusted positions of the reference elements resulting from the deforming match the relative positions of the reference elements of the measuring data with a sufficient accuracy. For example, the deforming may be executed iteratively, until a deviation of the adjusted positions of the reference elements from the relative positions of the reference elements reaches a predefined threshold.

For example, the method further comprises registering the preliminary three-dimensional digital jaw model with the measuring data. The registering comprises overlaying the preliminary three-dimensional digital jaw model and the measuring data. A relative position of the preliminary three-dimensional digital jaw model and the measuring data is adjusted until a mean deviation of the preliminary positions of reference elements described by the preliminary three-dimensional digital jaw model from the relative positions of the reference elements of the measuring data is minimized.

Examples may, e.g., comprise a registering of the preliminary three-dimensional digital jaw model with the measuring data. The registering may, e.g., in particular comprise a registering of the preliminary positions of the reference elements described by the preliminary three-dimensional digital jaw model with the relative positions of the reference elements of the measuring data. Based on this registering it may be determined, which deformations of the preliminary three-dimensional digital jaw model are necessary to adjust the preliminary positions of the reference elements such that the adjusted positions coincide with the relative positions of the measuring data, which are used as references for the deforming.

In the context of acquiring scan data of intraoral tissue, a registering may refer to a process of identifying and aligning digital scan data descriptive of surface structures of the intraoral tissue to create a representation of the patient's oral anatomy and/or the defining a relative position of the surface structures described by the scan data relative to a reference structure.

The acquired scan data may, e.g., comprise multiple data points of the soft and/or hard tissues of at least a part of a jaw within the oral cavity of the patient, such as, e.g., gingiva, alveolar ridges, and/or teeth. For example, multiple images representing multiple patches of a surface of the at least part of the jaw may be acquired. The captured data points and/or images may, e.g., be registered to ensure that they are accurately positioned relative to one another. This step may, e.g., involve matching overlapping regions of the scan to produce a continuous and coherent preliminary three-dimensional digital jaw model of the scanned at least part of the jaw. Once the registration is complete, the compiled scan data may, e.g., generate the preliminary three-dimensional digital jaw model of the scanned intraoral structures, e.g., the at least part of the jaw. This preliminary three-dimensional digital jaw model may, e.g., be used for treatment planning and/or for generating dental prostheses and/or appliances. In essence, registering may, e.g., ensure that acquired intraoral data is correctly aligned and usable for subsequent dental procedures or analyses.

For example, the adjusting of the relative position of the preliminary three-dimensional digital jaw model and the measuring data is executed iteratively. The relative position of the preliminary three-dimensional digital jaw model and the measuring data is, e.g., adjusted iteratively until a mean deviation of the preliminary positions of the reference elements in the preliminary three-dimensional digital jaw model from the relative positions of the reference elements of the measuring data reaches a predefined threshold.

For example, the receiving of the preliminary three-dimensional digital jaw model comprises generating the preliminary three-dimensional digital jaw model. The generating of the preliminary three-dimensional digital jaw model comprises receiving scan data of the at least part of the jaw. The scan data is used for the generating of the preliminary three-dimensional digital jaw model.

For example, the scan data comprises a plurality of sets of imaging data of a plurality of patches of a surface of the at least part of the jaw with the reference elements. The generating of the preliminary three-dimensional digital jaw model comprises assembling the imaging data of the plurality of patches of the surface of the at least part of the jaw.

For example, the assembling comprises a registering of the imaging data of adjacent patches of the plurality of patches with each other. The patches may, e.g., be assembled to provide a complete scan of the surface of the at least part of the jaw in form of a patchwork of the patches. In order to assemble the patches, they may be registered with each other. However, such a registering may comprise errors, which may add up. This may in particular be the case for a complex three-dimensional surface, like the surface of a jaw bow. For example, a jaw, e.g., a jaw bow, may not comprise or comprise only a few edges or similarly clearly distinguished structures, which may facilitate a registering.

For example, the method further comprises using pattern recognition for the registering of adjacent patches. The pattern recognition, e.g., comprises a two-dimensional pattern recognition. The pattern recognition, e.g., comprises a three-dimensional pattern recognition.

Pattern recognition refers to a process of identifying, analyzing, and interpreting patterns or features in scan data using computational algorithms and/or systems. A subset of pattern recognition is image pattern recognition which uses scan data provided in visual form, like images, for recognizing patterns. Key aspects of pattern recognition may comprise receiving an input comprising scan data; extracting features from the scan data, i.e., identifying specific characteristics or patterns within the scan data, such as, e.g., edges, textures, shapes, colors, or objects; analyzing the detected features, e.g., using an algorithm to classify, categorize, and/or interpret the detected features; and providing an output. The output may, e.g., comprise results of the pattern recognition, such as pattern identification, classification, segmentation, and/or anomaly detection. Pattern recognition may as well be used for identifying patterns in digital model. In case identical pattern are identified in two sets of scan data and/or in scan data and in a digital model and/or in two digital models, the identical pattern may, e.g., be used for a registering of the two sets of scan data and/or of the scan data and the digital model and/or of the two digital models, respectively.

Modern pattern recognition may, e.g., use machine learning and/or deep learning techniques, such as convolutional neural networks (CNNs), which may be configured to mimic human visual perception and achieve high levels of accuracy in recognizing complex patterns.

In case the patterns being recognized are two-dimensional patterns, the pattern recognition is referred to as a two-dimensional pattern recognition. In case the patterns being recognized are three-dimensional patterns, the pattern recognition is referred to as a three-dimensional pattern recognition.

For example, the scan data provides a higher local accuracy for determining surface structures of the individual surface patches compared to the measuring data, while the measuring data provides a higher global accuracy for determining the relative positions of the reference elements compared to the scan data.

Examples may provide the scan data in form of individual surface patches, which precisely resemble surface structures of the surface of the jaw. Using such scan data with individual surface patches providing a higher local accuracy for determining surface structures may ensure that the preliminary three-dimensional digital jaw model precisely resembles the surface structures of the surface of the at least part of the jaw. Deviations resulting from the patchwork character of the scan data may be compensated using the measuring data. The measuring data may provide a higher global accuracy for determining the relative positions of the reference elements compared to the scan data. Thus, the measuring data may be used to globally determine a precise distribution of the reference elements, e.g., implants, and their positions. This precisely determined global distribution of the reference elements and their positions may be used for correcting deviations of the preliminary three-dimensional digital jaw model resulting from the patchwork character of the scan data used for generating the preliminary three-dimensional digital jaw model. Thus, benefits of both the scanning method as well as the measuring method may be combined to provide an adjusted three-dimensional digital jaw model, which is more accurate than the preliminary three-dimensional digital jaw model and/or any three-dimensional digital jaw model, which may be generatable using only the measuring data.

For example, the scan data comprise optical scan data acquired using an optical scanning method.

For example, the optical scan data is acquired using an intraoral scan. For example, the optical scan data is acquired using a scan of an imprint of the at least part of the jaw. For example, the optical scan data is acquired using a scan of a physical model of the at least part of the jaw, e.g., manufactured using an imprint.

Optical scanning methods, in particular intraoral optical scanning methods, may be configured to provide scan data, which resembles local surface structures of the surface of the at least part of the jaw with a high accuracy. However, the scan data may provide imaging data of the surface of the at least part of the jaw in form of a plurality of patches of the surface. Thus, in order to generate a three-dimensional digital jaw model using the scan data assembling of the patches may be required. In order to correct potential deviations, which may arise from the assembling, measuring data may be used.

Optical scanning, in particular intraoral optical scanning, refers to a digital imaging technique used in dentistry to capture detailed three-dimensional representations of a patient's intraoral structures, including gingiva, implants, abutments, teeth, and other intraoral structures and/or oral tissues. This is achieved using a scanning device, e.g., a handheld device, equipped with one or more light sources and one or more optical sensors. The scanning device may emit light, which is projected onto a surface to be scanned, e.g., on a surface inside an oral cavity. Multiple high-resolution images or patches of imaging data are captured from different angles as the scanning device is moved over the surface to be scanned. The images are processed by software, i.e., the images or patches of imaging data are assembled, to generate detailed three-dimensional digital model of the scanned surface.

For example, the scan data comprise X-ray scan data acquired using an X-ray based imaging method, i.e., an X-ray based scanning method. The X-ray scan data may, e.g., be acquired using the X-ray based imaging method for directly scanning the oral cavity of the patient with the reference elements, e.g., implants, arranged therein.

For example, the X-ray based imaging method is an X-ray based three-dimensional imaging method, e.g., CT or CBCT. Computed Tomography (CT) refers to a medical imaging technique that uses X-rays to create cross-sectional images of anatomic structures. It may involve rotating an X-ray source and detector around the patient to capture multiple two-dimensional images from different angles. These images may then be reconstructed by a computer to generate a three-dimensional representation of the scanned structure. CT may be able to provide comprehensive details of both hard and soft tissues.

Cone Beam Computed Tomography (CBCT) refers to a specialized form of CT imaging designed specifically for dental and maxillofacial applications. Unlike traditional CT, which uses a fan-shaped beam, CBCT uses a cone-shaped X-ray beam that may capture an entire volume of interest in a single rotation. CT may offer a higher resolution for soft tissue imaging, while CBCT may excel in hard tissue imaging compared to CT, such as teeth and bones.

For example, the measuring data comprise measuring data of at least a section of the at least part of the jaw comprising all the reference elements, e.g., implants. Example may enable an adjusting of the preliminary three-dimensional digital jaw model using the measuring data, which may cover all the reference elements and thus provide a complete description of the relative positions of the reference elements arranged within the at least part of the jaw.

For example, the reference elements may be implants arranged within the at least part of the jaw. Using implants and their relative positions, as provided by the measuring data, as reference elements, i.e., reference markers, for the adjusting, i.e., deforming, of the preliminary three-dimensional digital jaw model may, e.g., be beneficial, since the implants may be characteristic structures within the at least part of the jaw and in particular with the flexible gingiva of the at least part of the jaw, which may be precisely locatable. Furthermore, a locating of the implants and their positions may be further facilitated using physical markers, like abutments and/or dedicated physical fiducial markers, which may be arranged within the implants and indicate their positions.

For example, the measuring method used for acquiring the measuring data is a second scanning method. For example, the measuring data are provided in form of second scan data.

For example, the measuring data comprise optical measuring data acquired using an optical measuring method. The optical measuring method may, e.g., be different from the optical scanning method. The optical measuring method may be configured to provide optical measuring data, e.g., imaging data, which at least cover a section of the at least part of the jaw, which comprises all the reference elements and which is not divided into patches. The measuring data may rather provide a single image or imaging patch, which covers at least the respective section. The measuring data may provide overlapping images or imaging patches, which each cover at least the section of the at least part of the jaw comprising all the implants.

A difference between the scan data and measuring data may, e.g., be that the patches of the scan data may only overlap at boundary sections. The overlapping boundary sections of the patches of the patches of the scan data may, e.g., be used for an assembling of the patches. The patches are assembled to extend a coverage of the surface of the at least part of the jaw being depicted by the scan data.

The measuring data may comprise patches depicting the section of the at least part of the jaw comprising all the reference elements from different directions. The overlapping sections of the measuring data are, e.g., used for determining spatial information, in particular a measurement of distances of the reference elements from the sensors used for acquiring the measuring data. The overlap is not used for extending a coverage of the surface of the at least part of the jaw, but rather to acquire additional information regarding the overlapping sections, i.e., the section of the at least part of the jaw comprising all the reference elements. The additional information may, e.g., be the spatial information, in particular the distances of the reference elements from the sensors used for acquiring the measuring data.

For example, the optical measuring method comprises an evaluation using a photogrammetric analysis of the optical measuring data being acquired. For example, a photogrammetric optical measuring method is used for acquiring the optical measuring data. For example, stereophotogrammetry is used.

A photogrammetric analysis refers to a method for obtaining measurements and spatial information about surfaces and/or objects from optical imaging data, like optical images, and/or other data imaging data. It may comprise an analyzing two-dimensional imaging data to reconstruct three-dimensional digital models and/or determine a shape, size, position, and/or other geometric properties of the surfaces and/or objects being scanned. Key features a photogrammetric analysis may comprise a usage of overlapping two-dimensional imaging data acquired from different angles. Here, the overlap may be used to acquire additional information, e.g., regarding distances of the surface and/or objects. It may, e.g., use triangulation to determine relative positions of points in the three-dimensional space.

Stereophotogrammetry is a specialized form of photogrammetry that uses two or more synchronized optical sensors, e.g., cameras, to acquire imaging data of a surface and/or an object from different viewpoints simultaneously. These acquired imaging data acquired from different viewpoints simultaneously may then be processed to reconstruct a three-dimensional representation, e.g., a three-dimensional digital model of the scanned surface with high accuracy. For the stereophotogrammetry optical sensors arranged at known fixed relative spatial positions relative to each other. Scan data in form of optical imaging data may be acquired simultaneously using the optical sensors arranged at the fixed relative spatial positions relative to each other. For example, corresponding points between the scan data acquired by the different sensors may be determined and their positions may be calculated in the three-dimensional space. For this calculation triangulation may be used based on the known fixed relative spatial positions of the sensors relative to each other.

Photogrammetric analysis focuses generally on extracting measurements and three-dimensional information from two-dimensional imaging data. Stereophotogrammetry refers to a subset that specifically uses multiple imaging data taken simultaneously to enhance accuracy.

For example, the measuring data comprise tactile measuring data acquired using a tactile measuring method.

For example, the measuring data is acquired using a tactile scan, which is an extraoral tactile scan. For example, the measuring data is acquired using a tactile scan of an imprint of the at least part of the jaw. For example, the measuring data is acquired using a tactile scan of a physical model of the at least part of the jaw, e.g., manufactured using an imprint. For example, the measuring data is acquired using an intraoral tactile scan.

A tactile measuring method refers to a technique for capturing a three-dimensional structure of intraoral tissues, like at least a part of a jaw, or dental models by physically contacting the surface of the object being scanned. This method may involve the use of a mechanical and/or digital probe that traces contours of hard and/or soft tissues of the intraoral tissue or a model thereof, recording their shape and/or dimensions. Key features of a tactile measuring method may, e.g., comprise a physical contact. A probe, e.g., a stylus and/or sensor, may come into direct contact with a surface to be scanned for measuring its shape and/or geometric features. A point-by-point measurement or scan may be expected. The probe may, e.g., be moved systematically across the respective surface, collecting scan data at multiple points of the surface to create a three-dimensional digital representation of the surface being scanned.

For example, the measuring data comprise X-ray measuring data acquired using an X-ray based imaging method. The X-ray measuring data may, e.g., be acquired using the X-ray based imaging method for directly scanning the oral cavity of the patient with the reference elements, e.g., implants, arranged therein.

For example, the X-ray based imaging method is an X-ray based three-dimensional imaging method, e.g., CT or CBCT. Computed Tomography (CT) refers to a medical imaging technique that uses X-rays to create cross-sectional images of anatomic structures. It may involve rotating an X-ray source and detector around the patient to capture multiple two-dimensional images from different angles. These images may then be reconstructed by a computer to generate a three-dimensional representation of the scanned structure. CT may be able to provide comprehensive details of both hard and soft tissues.

Cone Beam Computed Tomography (CBCT) refers to a specialized form of CT imaging designed specifically for dental and maxillofacial applications. Unlike traditional CT, which uses a fan-shaped beam, CBCT uses a cone-shaped X-ray beam that may capture an entire volume of interest in a single rotation. CT may offer a higher resolution for soft tissue imaging, while CBCT may excel in hard tissue imaging compared to CT, such as teeth and bones.

For example, the measuring data comprise magnetic resonance imaging (MRI) data acquired using an MRI method. The MRI data may, e.g., be acquired using the MRI method for directly scanning the oral cavity of the patient with the reference elements, e.g., implants, arranged therein.

Magnetic Resonance Imaging (MRI) refers to a non-invasive imaging technique that uses strong magnetic fields, radio waves, and computer technology to create detailed images of the internal anatomic structures of a patient. MRI uses a strong magnetic field for aligning hydrogen atoms in the body. Pulses of radio waves are used to disrupt this alignment, causing the hydrogen atoms to emit signals as they return to their original state. A computer may process these signals to generate detailed 2D or 3D images of tissues and anatomic structures.

For example, the reference elements comprise implants arranged within the at least part of the jaw.

For example, physical markers arranged on the implants are used for determining the relative positions of the reference elements, i.e., implants, within the measuring data. Examples may facilitate the determining of the relative positions of the implants using the physical markers. The physical markers may be easier to recognize than the implants themselves. This may, e.g., be due to the fact that the physical markers protrude from the implants, on which they may be arranged. Furthermore, the physical markers may have physical forms and/or surfaces which are easier to recognize than forms and/or surfaces of the implants. The physical forms and/or surfaces of the physical markers may, e.g., be clearly distinguishable from the form and/or surface of the at least part of the jaw, in particular from the form and/or surface of a gingiva of the at least part of the jaw. With the physical markers easier detectable, their positions and thus the positions of the implants, on which they are arranged, may be easier detectable compared to scan data without scan data of the physical markers.

For example, the physical markers may only be applied, while acquiring the measuring data. For example, the physical markers may only be applied, while acquiring the scan data.

For example, the physical markers may be applied, while acquiring the scan data and the measuring data. For example, the physical markers arranged on the implants may be further used for determining the preliminary implant positions within the preliminary three-dimensional digital jaw model.

For example, the physical markers comprise abutments. Abutments may protrude from the implants, on which they may be arranged, and may thus be easier detectable than the implants themselves. Furthermore, the abutments may, e.g., have physical forms and/or surfaces which are clearly distinguishable from the form and/or surface of the at least part of the jaw, in particular from the form and/or surface of a gingiva of the at least part of the jaw. Thus, the abutments may be easier recognizable than implants, which are arranged within the respective jaw, e.g., jaw bow, rather than on the same.

For example, the physical markers comprise dedicated physical fiducial markers with predefined physical forms and surfaces. A dedicated physical fiducial marker refers to a specifically configured reference object used to assist in an accurate localization and/or registration of scan data in a spatial context. Fiducial markers may, e.g., further usable to establish a common coordinate system between the physical world and the digital environment. A fiducial marker may be a tangible object, e.g., affixed to a reference position of a surface. A fiducial marker may be configured for a clear visibility and distinctiveness for a scanning method. It may, e.g., comprise contrasting colors, unique shapes, and/or coded patterns. Geometric properties of a fiducial marker may be known. Dimensions, shape, and/or location of the marker may, e.g., be precisely known, making it a reliable reference point. A dedicated fiducial marker may be specifically tailored and/or optimized for a given application, such as a dental scanning method.

Dedicated physical fiducial markers may protrude from the implants, on which they may be arranged, and may thus be easier detectable than the implants themselves. Furthermore, the dedicated physical fiducial markers may, e.g., have physical forms and/or surfaces which are clearly distinguishable from the form and/or surface of the at least part of the jaw, in particular from the form and/or surface of a gingiva of the at least part of the jaw. Thus, the dedicated physical fiducial markers may be easier recognizable than implants, which are arranged within the respective jaw, e.g., jaw bow, rather than on the same.

For example, the deforming comprises using a Laplacian surface editing. Laplacian surface editing refers to a technique used in computer graphics and computational geometry to modify and manipulate three-dimensional digital surfaces while preserving local geometric details. The method leverages the Laplacian operator, which captures a local curvature or differential properties of a surface, allowing for smooth and natural edits. Key features of Laplacian surface editing may comprise a preservation of local details. The Laplacian operator encodes differences between individual vertices and their vertices in a mesh-structure defining the surface to be edited, e.g., deformed. This may ensure that when the surface is edited, e.g., deformed, local geometric details, such as curvature and fine features, may be preserved. Laplacian surface editing may, e.g., further comprise a constraint-based editing. A user may impose one or more constraints on specific points or regions of the surface, such as fixing certain vertices or specifying new positions for them. The method may then adjust surrounding surface section to smoothly accommodate these changes. The specifying of new positions of vertices may, e.g., comprise an adjusting of the preliminary positions of the reference elements to coincide with the relative positions of the reference elements used as references for the deforming. Surface sections of the preliminary three-dimensional digital jaw model may then be smoothly accommodated to these adjustments of the preliminary positions of the reference elements resulting in a deformation of the preliminary three-dimensional digital jaw model, which may preserve local geometric details, such as curvature and fine features, of the preliminary three-dimensional digital jaw model. Laplacian surface editing may, e.g., implement smooth transitions. Usage of the Laplacian operator may, e.g., ensure that changes propagate naturally across the surface of the preliminary three-dimensional digital jaw model, generating smooth deformations and avoiding abrupt transitions.

Laplacian surface editing may, e.g., allow for modifying the preliminary three-dimensional digital jaw model without losing anatomical accuracy. Laplacian surface editing may, e.g., ensure a high-fidelity maintaining authenticity of original surface features; flexibility supports a wide range of potential editing scenarios from minor adjustments to significant deformations; and computational efficiency.

For example, a Laplacian surface editing as described in “Laplacian surface editing” by Sorkine, O. et al., in Proceedings of the 2004 Eurographics/ACM SIGGRAPH Symposium on Geometry Processing, 2004, pages 175 to 184 may be used. For performing surface editing operations geometric details to be preserved as much as possible of a surface of a structure to be deformed may be determined. Thus, when executing the surface editing operations for deforming the respective structure, a resulting deformed structure may still resemble characteristic features in form of the preserved geometric details, such that the resulting structure is recognizable as a deformed version of the original structure. Surface editing may, e.g., be performed by operating over an intrinsic surface representation, which may be provided based on a Laplacian of a mesh representing the surface by encoding each vertex of the mesh relative to its neighborhood. The Laplacian of the mesh may be enhanced to be invariant to locally linearized rigid transformations and scaling. Using such a Laplacian representation, surface editing operations may be defined, e.g., comprising interactive free-form deformation in a region of interest based on a transformation of a handle.

The preliminary three-dimensional digital jaw model may, e.g., be provided by a three-dimensional digital mesh structure, which is adjusted to the reference positions described by the measuring data using a mesh deformation. With the preliminary three-dimensional digital jaw model being provided by a three-dimensional digital mesh structure also the adjusted three-dimensional digital jaw model resulting from the deforming of the preliminary three-dimensional digital jaw model is provided by a three-dimensional digital mesh structure, i.e., an adjusted three-dimensional digital mesh structure. With a three-dimensional digital mesh structure representing the surface of the preliminary three-dimensional digital jaw model a Laplacian surface editing may comprise a Laplacian editing of the mesh of the mesh structure representing the surface of the preliminary three-dimensional digital jaw model.

For example, the method further comprises generating a three-dimensional digital prosthesis model of an implant-based dental prosthesis using the adjusted three-dimensional digital jaw model.

The three-dimensional digital prosthesis model may, e.g., be generated such that it comprises a support surface configured as a negative of at least a section of the surface of the at least part of the jaw resembled by the adjusted three-dimensional digital jaw model. Thus, the support surface may accurately fit onto the respective section of the surface of the at least part of the jaw.

For example, the three-dimensional digital prosthesis model comprises receptions for receiving fastening elements configured for fastening the implant-based dental prosthesis to implants, e.g., to implants used as reference elements for the adjusting of the preliminary three-dimensional digital jaw model.

Positions and/or orientations of the receptions within the three-dimensional digital prosthesis model may, e.g., be determined using positions and/or orientations of implants defined by the adjusted three-dimensional digital jaw model, i.e., the three-dimensional digital jaw model resulting from the adjusting of the preliminary three-dimensional digital jaw model. The receptions may be configured such that they are aligned with the implants, when the three-dimensional digital prosthesis model is arranged on the adjusted three-dimensional digital jaw model. Furthermore, e.g., sizes and/or forms of the fastening elements may be used for determining, e.g., sizes and/or forms of the reception within the three-dimensional digital prosthesis model.

The fastening elements comprising one or more of the following: abutments, screws.

For example, the implant-based dental prosthesis is an implant-based dental bridge. For example, the implant-based dental bridge may be a bridge comprising a full dental arch, i.e., an implant-based denture. The bridge may, e.g., comprise a maxillary dental arch, i.e., a maxillary implant-based denture may be provided. The bridge may, e.g., comprise a mandibular dental arch, i.e., a mandibular implant-based denture may be provided.

For example, the method further comprises using the three-dimensional digital prosthesis model as a template for controlling a manufacturing device to manufacture the implant-based dental prosthesis as a physical copy of the template.

Examples may, e.g., provide an implant-based dental prosthesis as a physical copy of the three-dimensional digital prosthesis model. With the receptions of the three-dimensional digital prosthesis model precisely adjusted to the implant positions and/or orientations as defined by the adjusted three-dimensional digital jaw model, an accurate fitting of the implant-based dental prosthesis onto the implants may, e.g., be ensured. With a form of the three-dimensional digital prosthesis model precisely adjusted to a surface of the at least part of the jaw as described by the adjusted three-dimensional digital jaw model resulting from using a combination of the scan data and measuring data, an accurate fitting of the implant-based dental prosthesis onto the at least part of the jaw may, e.g., be ensured.

For example, a method for adjusting a preliminary three-dimensional digital jaw model of a jaw bow of a patient is disclosed. The method comprises receiving scan data acquired using a scanning method. The scan data comprise a plurality of sets of imaging data of a plurality of patches of a surface of the jaw bow with implants arranged within the jaw bow. A preliminary three-dimensional digital jaw model is generated. The generating comprises assembling the imaging data of the plurality of patches of the surface of the jaw bow. The implants represented by the preliminary three-dimensional digital jaw model have preliminary implant positions. Measuring data, e.g., in form of second scan data, is acquired using a measuring method, e.g., a second scanning method, are received. The measuring data is descriptive of relative positions of the implants arranged within the jaw bow. The preliminary three-dimensional digital jaw model is deformed. The deforming results in an adjusted three-dimensional digital jaw model with adjusted implant positions using the relative positions of the implants as references, i.e., reference markers, for the deforming.

In another aspect, a computer program product for adjusting a preliminary three-dimensional digital jaw model of at least a part of a jaw of a patient is disclosed. The computer program product comprises a non-transitory computer-readable storage medium having program instructions embodied therewith. The program instructions are executable by a processing unit of a computer device to cause the processing unit to control the computer device to receive the preliminary three-dimensional digital jaw model of the at least part of the jaw. The preliminary three-dimensional digital jaw model is descriptive of preliminary positions of reference elements of the jaw. Measuring data of an additional measurement of relative positions of the reference elements of the jaw are received. The preliminary three-dimensional digital jaw model is deformed. The deforming results in an adjusted three-dimensional digital jaw model with adjusted positions of the reference elements using the measured relative positions of the reference elements described by the measuring data as references for the deforming.

Execution of the program instructions by a processing unit of a computer device may, e.g., cause the computer device to execute any of the aforementioned examples of the method for adjusting a preliminary three-dimensional digital jaw model of at least a part of a jaw of a patient.

For example, a computer program for generating a three-dimensional digital jaw model of at least a part of a jaw of a patient is provided. The computer program comprises program instructions, which are executable by a processing unit of a computer device to cause the processing unit to control the computer device to receive the preliminary three-dimensional digital jaw model of the at least part of the jaw. The preliminary three-dimensional digital jaw model is descriptive of preliminary positions of reference elements of the jaw. Measuring data of an additional measurement of relative positions of the reference elements of the jaw are received. The preliminary three-dimensional digital jaw model is deformed. The deforming results in an adjusted three-dimensional digital jaw model with adjusted positions of the reference elements using the measured relative positions of the reference elements described by the measuring data as references for the deforming.

Execution of the program instructions by a processing unit of a computer device may, e.g., cause the computer device to execute any of the aforementioned examples of the method for adjusting a preliminary three-dimensional digital jaw model of at least a part of a jaw of a patient.

In another aspect, a computer device for generating a three-dimensional digital jaw model of at least a part of a jaw of a patient is disclosed. The computer device comprises a processing unit and a memory unit storing program instructions executable by the processing unit. An execution of the program instructions by the processing unit causes the processing unit to control the computer device to receive the preliminary three-dimensional digital jaw model of the at least part of the jaw. The preliminary three-dimensional digital jaw model is descriptive of preliminary positions of reference elements of the jaw. Measuring data of an additional measurement of relative positions of the reference elements of the jaw are received. The preliminary three-dimensional digital jaw model is deformed. The deforming results in an adjusted three-dimensional digital jaw model with adjusted positions of the reference elements using the measured relative positions of the reference elements described by the measuring data as references for the deforming.

Execution of the program instructions by the processing unit of the computer device may, e.g., cause the computer device to execute any of the aforementioned examples of the method for adjusting a preliminary three-dimensional digital jaw model of at least a part of a jaw of a patient.

For example, the computer device is comprised by a manufacturing system. The computer device may be any of the aforementioned examples of a computer device. The manufacturing system may further comprise one or more manufacturing devices configured to manufacture an implant-based dental prosthesis.

Execution of the program instructions by the processing unit further causes the computer device to generate a three-dimensional digital prosthesis model of the implant-based dental prosthesis using the three-dimensional digital jaw model. The three-dimensional digital prosthesis model comprises receptions for receiving fastening elements configured for fastening the implant-based dental prosthesis to implants. Data for controlling the manufacturing of the implant-based dental prosthesis, i.e., a physical implant-based dental prosthesis, is provided. The data defines the three-dimensional digital prosthesis model as a template for the physical implant-based dental prosthesis. The one or more manufacturing devices are controlled to manufacture the physical implant-based dental prosthesis using the data provided for controlling the manufacturing with the manufactured physical implant-based dental prosthesis being a physical copy of the template defined by the data provided.

For the manufacturing of the implant-based dental prosthesis, e.g., computer-controlled additive and/or subtractive methods may be used. For example, the implant-based dental prosthesis and/or elements of the implant-based dental prosthesis may be manufactured using one of the following: machining, 3D printing, casting.

For example, the one or more manufacturing devices of the manufacturing system may comprise one or more of the following: a machining device, a 3D printing device.

It is understood that one or more of the aforementioned examples may be combined as long as the combined examples are not mutually exclusive.

### BRIEF DESCRIPTION OF THE DRAWINGS

In the following, examples are described in greater detail making reference to the drawings in which:

FIG. 1 is a flowchart of an exemplary method for adjusting a preliminary three-dimensional digital jaw model of at least a part of a jaw of a patient;

FIG. 2 is a flowchart of an exemplary method for adjusting a preliminary three-dimensional digital jaw model of at least a part of a jaw of a patient;

FIG. 3 is a flowchart of an exemplary method for adjusting a preliminary three-dimensional digital jaw model of at least a part of a jaw of a patient;

FIG. 4 is a flowchart of an exemplary method for adjusting a preliminary three-dimensional digital jaw model of at least a part of a jaw of a patient;

FIG. 5 is a flowchart of an exemplary method for adjusting a preliminary three-dimensional digital jaw model of at least a part of a jaw of a patient;

FIG. 6 is a flowchart of an exemplary method for adjusting a preliminary three-dimensional digital jaw model of at least a part of a jaw of a patient;

FIG. 7 shows an exemplary preliminary three-dimensional digital jaw model being deformed;

FIG. 8 shows exemplary adjusted three-dimensional digital jaw models of a maxilla and a mandible;

FIG. 9 shows exemplary three-dimensional digital prosthesis models of implant-based dental prostheses;

FIG. 10 shows an exemplary oral cavity with a jaw bow;

FIG. 11 shows an exemplary implant-based dental prosthesis;

FIG. 12 shows a schematic diagram of an exemplary computer device for generating a three-dimensional digital jaw model of a jaw bow of a patient;

FIG. 13 shows a schematic diagram of an exemplary system comprising a computer device; and

FIG. 14 shows a schematic diagram of an exemplary manufacturing system for manufacturing an implant-based dental prosthesis.

### DETAILED DESCRIPTION

In the following, similar elements are denoted by the same reference numerals.

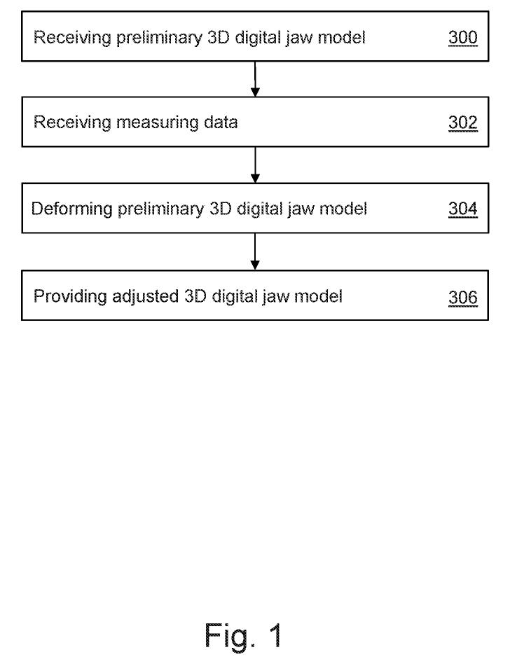

FIG. 1 shows an exemplary method for adjusting a preliminary three-dimensional digital jaw model of at least a part of a jaw of a patient is disclosed. In block 300, the preliminary three-dimensional digital jaw model of the at least part of the jaw is received. The preliminary three-dimensional digital jaw model is descriptive of preliminary positions of reference elements of the jaw, e.g., implants arranged within the jaw. In block 302, measuring data of an additional measurement of relative positions of the reference elements of the jaw are received. In block 304, the preliminary three-dimensional digital jaw model is deformed. The deforming results in an adjusted three-dimensional digital jaw model with adjusted positions of the reference elements using the measured relative positions of the reference elements described by the measuring data as references for the deforming. In block 306, the resulting adjusted three-dimensional digital jaw model is provided.

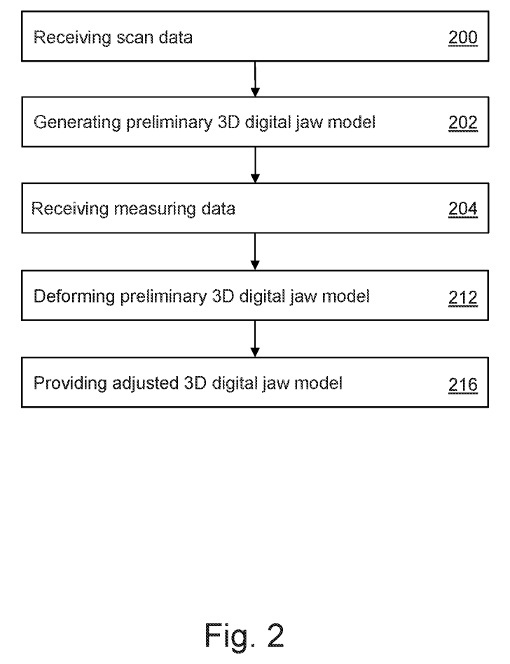

FIG. 2 shows an exemplary method for adjusting a preliminary three-dimensional digital jaw model of at least a part of a jaw of a patient. The at least part of the jaw, e.g., is an edentulous part of the jaw. The at least part of the jaw, e.g., is at least a part of an edentulous jaw bow. In block 200, scan data acquired using a scanning method are received. The scan data comprise a plurality of sets of imaging data of a plurality of patches of a surface of the at least part of the jaw with reference elements, e.g., implants, arranged within the at least part of the jaw. For example, the scan data comprise optical scan data acquired using an optical scanning method. For example, the optical scan data is acquired using an intraoral scan. For example, the optical scan data is acquired using a scan of an imprint of the at least part of the jaw. For example, the optical scan data is acquired using a scan of a physical model of the at least part of the jaw manufactured, e.g., using an imprint.

In block 202, a preliminary three-dimensional digital jaw model is generated. The generating comprises assembling the imaging data of the plurality of patches of the surface of the at least part of the jaw. The assembling, e.g., comprises a registering of the imaging data of adjacent patches of the plurality of patches with each other. For example, the method further comprises using pattern recognition for the registering of adjacent patches. The pattern recognition, e.g., comprises a two-dimensional pattern recognition. The pattern recognition, e.g., comprises a three-dimensional pattern recognition. For example, the preliminary three-dimensional digital jaw model is descriptive of preliminary positions of the reference elements. For example, reference elements represented by the preliminary three-dimensional digital jaw model have preliminary positions.

In block 204, measuring data acquired using a measuring method are received. The measuring data is descriptive of relative positions of the reference elements arranged within the at least part of the jaw. For example, the measuring data comprise measuring data of at least a section of the at least part of the jaw comprising all the reference elements. For example, the scan data provides a higher local accuracy for determining surface structures of the individual surface patches compared to the measuring data, while the measuring data providing a higher global accuracy for determining the relative positions of the reference elements compared to the scan data.

For example, the measuring data comprise optical measuring data acquired using an optical measuring method. For example, the optical measuring data comprised by the measuring data is acquired using an optical measuring method, which uses a photogrammetric analysis for evaluating the optical measuring data being acquired. For example, stereophotogrammetry is used. For example, the measuring data comprise tactile measuring data acquired using a tactile measuring method. For example, the measuring data comprise X-ray measuring data acquired using an X-ray based imaging method. For example, the X-ray based imaging method is an X-ray based three-dimensional imaging method, e.g., CT or CBCT. For example, the reference elements are implants. For example, physical markers arranged on the implants are used for determining the relative positions of the implants within the measuring data. For example, the physical markers comprise abutments. For example, the physical markers comprise dedicated physical fiducial markers with predefined physical forms and surfaces.

In block 212, the preliminary three-dimensional digital jaw model is deformed. The deforming results in an adjusted three-dimensional digital jaw model with adjusted positions of the reference elements using the relative positions of the reference elements as references for the deforming. For example, the deforming comprises using a Laplacian surface editing. The three-dimensional digital jaw model is, e.g., provided by a three-dimensional digital mesh structure, which is adjusted to the reference positions described by the measuring data using a mesh deformation. In block 216, the resulting adjusted three-dimensional digital jaw model is provided.

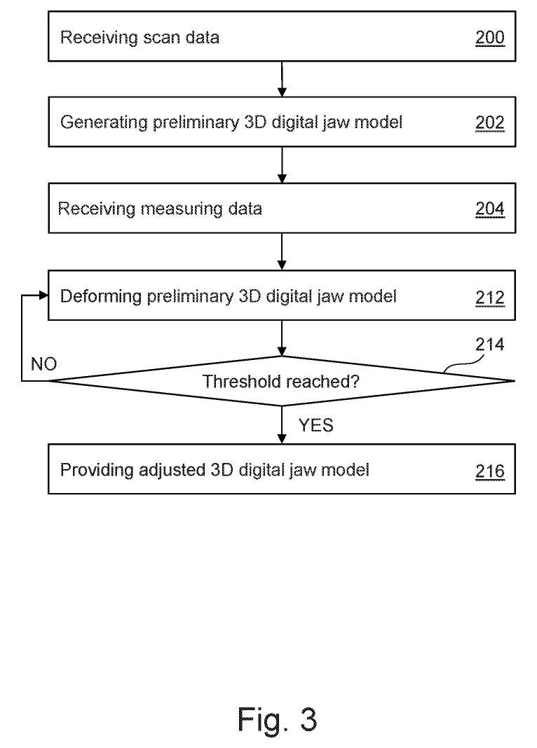

FIG. 3 shows an exemplary method for adjusting a preliminary three-dimensional digital jaw model of at least a part of a jaw of a patient. In block 200, scan data acquired using a scanning method are received. The scan data comprise a plurality of sets of imaging data of a plurality of patches of a surface of the at least part of the jaw with reference elements, e.g., implants, arranged within the at least part of the jaw. In block 202, a preliminary three-dimensional digital jaw model is generated. The generating comprises assembling the imaging data of the plurality of patches of the surface of the at least part of the jaw. For example, the preliminary three-dimensional digital jaw model is descriptive of preliminary positions of the reference elements. For example, reference elements represented by the preliminary three-dimensional digital jaw model have preliminary positions. In block 204, measuring data acquired using a measuring method are received. The measuring data is descriptive of relative positions of the reference elements arranged within the at least part of the jaw. In block 212, the preliminary three-dimensional digital jaw model is deformed. The deforming results in an adjusted three-dimensional digital jaw model with adjusted positions of the reference elements using the relative positions of the reference elements as references for the deforming. The deforming may, e.g., be executed iteratively until the adjusted positions of the reference elements resulting from the deforming match the relative positions of the reference elements with a sufficient accuracy. For example, it is checked in block 214 whether a predefined threshold for the accuracy is reached. If the threshold is not reached, the deforming may be continued in block 212. If the threshold is reached, the deforming may be terminated and the resulting three-dimensional digital jaw model provided in block 216.

Blocks 200, 202, 204, 212, and 216 of FIG. 3 may, e.g., correspond to blocks 200, 202, 204, 212, and 216 of FIG. 2. Thus, the features disclosed in the context of the method of FIG. 2 may also be comprised by and/or applied to the method of FIG. 3.

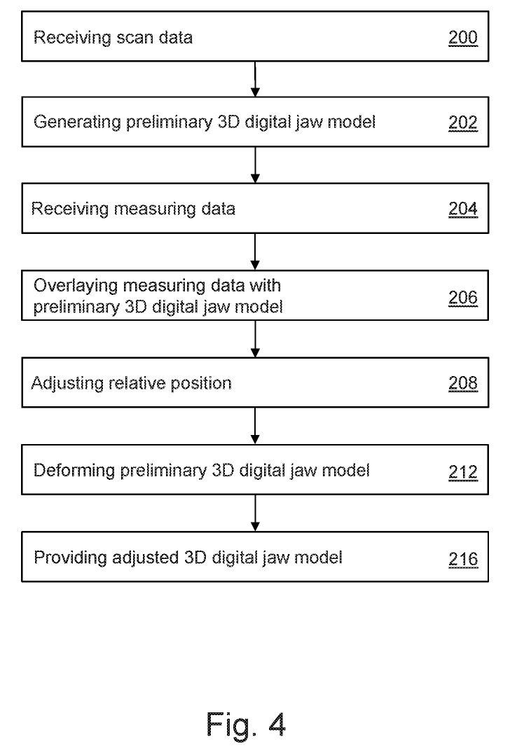

FIG. 4 shows an exemplary method for adjusting a preliminary three-dimensional digital jaw model of at least a part of a jaw of a patient. In block 200, scan data acquired using a scanning method are received. The scan data comprise a plurality of sets of imaging data of a plurality of patches of a surface of the at least part of the jaw with reference elements, e.g., implants, arranged within the at least part of the jaw. In block 202, a preliminary three-dimensional digital jaw model is generated. The generating comprises assembling the imaging data of the plurality of patches of the surface of the at least part of the jaw. For example, the preliminary three-dimensional digital jaw model is descriptive of preliminary positions of the reference elements. For example, reference elements represented by the preliminary three-dimensional digital jaw model have preliminary positions. In block 204, measuring data acquired using a measuring method are received. The measuring data is descriptive of relative positions of the reference elements arranged within the at least part of the jaw.

In block 206, the preliminary three-dimensional digital jaw model and the measuring data are overlaid. A purpose of the overlaying is, e.g., a registering the preliminary three-dimensional digital jaw model with the measuring data. In block 208 a relative position of the preliminary three-dimensional digital jaw model and the measuring data is adjusted. By adjusting the relative position of the preliminary three-dimensional digital jaw model relative to the measuring data the positions of the reference elements in the preliminary three-dimensional digital jaw model may be aligned with the positions of the reference elements in the measuring data. For example, the relative position is adjusted until a mean deviation of the preliminary positions of the reference elements in the preliminary three-dimensional digital jaw model from the relative positions of the reference elements in the measuring data is minimized.

In block 212, the preliminary three-dimensional digital jaw model is deformed. The deforming results in an adjusted three-dimensional digital jaw model with adjusted positions of the reference elements using the relative positions of the reference elements as references for the deforming. In block 216, the resulting adjusted three-dimensional digital jaw model is provided.

Blocks 200, 202, 204, 212, and 216 of FIG. 4 may, e.g., correspond to blocks 200, 202, 204, 212, and 216 of FIG. 2 and/or FIG. 3. Thus, the features disclosed in the context of the method of FIGS. 2 and/or 3 may also be comprised by and/or applied to the method of FIG. 4.

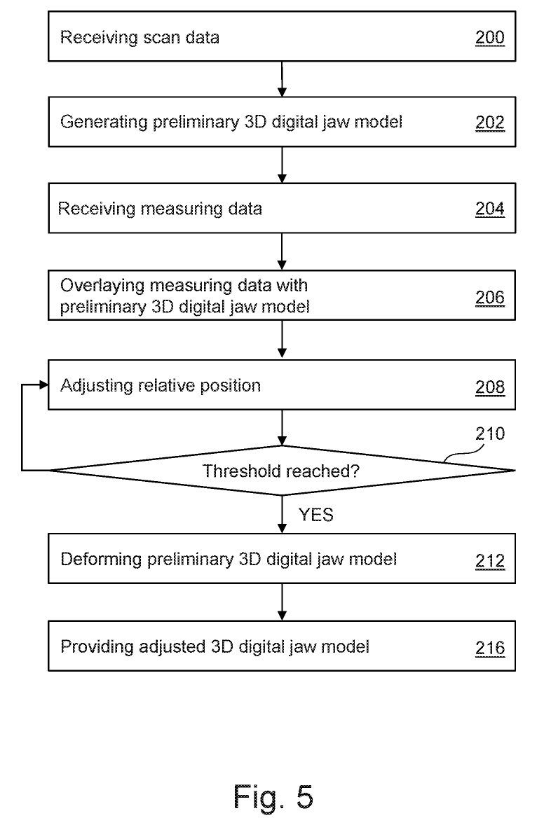

FIG. 5 shows an exemplary method for adjusting a preliminary three-dimensional digital jaw model of at least a part of a jaw of a patient. In block 200, scan data acquired using a scanning method are received. The scan data comprise a plurality of sets of imaging data of a plurality of patches of a surface of the at least part of the jaw with reference elements, e.g., implants, arranged within the at least part of the jaw. In block 202, a preliminary three-dimensional digital jaw model is generated. The generating comprises assembling the imaging data of the plurality of patches of the surface of the at least part of the jaw. For example, the preliminary three-dimensional digital jaw model is descriptive of preliminary positions of the reference elements. For example, reference elements represented by the preliminary three-dimensional digital jaw model have preliminary positions. In block 204, measuring data acquired using a measuring method are received. The measuring data is descriptive of relative positions of the reference elements arranged within the at least part of the jaw.

In block 206, the preliminary three-dimensional digital jaw model and the measuring data are overlaid. A purpose of the overlaying is, e.g., a registering the preliminary three-dimensional digital jaw model with the measuring data. In block 208 a relative position of the preliminary three-dimensional digital jaw model and the measuring data is adjusted. By adjusting the relative position of the preliminary three-dimensional digital jaw model relative to the measuring data the positions of the reference elements in the preliminary three-dimensional digital jaw model may be aligned with the positions of the reference elements in the measuring data. For example, the relative position is adjusted until a mean deviation of the preliminary positions of the reference elements in the preliminary three-dimensional digital jaw model from the relative positions of the reference elements in the measuring data is minimized.

The adjusting of the relative position of the preliminary three-dimensional digital jaw model and the measuring data may, e.g., be executed iteratively. For example, a minimization approach may be used. In block 210 it is checked whether a threshold for the mean deviation of the preliminary positions of the reference elements in the preliminary three-dimensional digital jaw model from the relative positions of the reference elements in the measuring data is reached. If the threshold is not reached, the adjusting of the relative position may be continued in block 208. If the threshold is reached, the adjusting may be terminated and the method continued in block 212 using the resulting relative position of the preliminary three-dimensional digital jaw model and the measuring data.

In block 212, the preliminary three-dimensional digital jaw model is deformed. The deforming results in an adjusted three-dimensional digital jaw model with adjusted positions of the reference elements using the relative positions of the reference elements as references for the deforming. In block 216, the resulting adjusted three-dimensional digital jaw model is provided.

Blocks 200, 202, 204, 212, and 216 of FIG. 5 may, e.g., correspond to blocks 200, 202, 204, 212, and 216 of any of FIGS. 2-4. Thus, the features disclosed in the context of the method of any of FIGS. 2-4 may also be comprised by and/or applied to the method of FIG. 5.

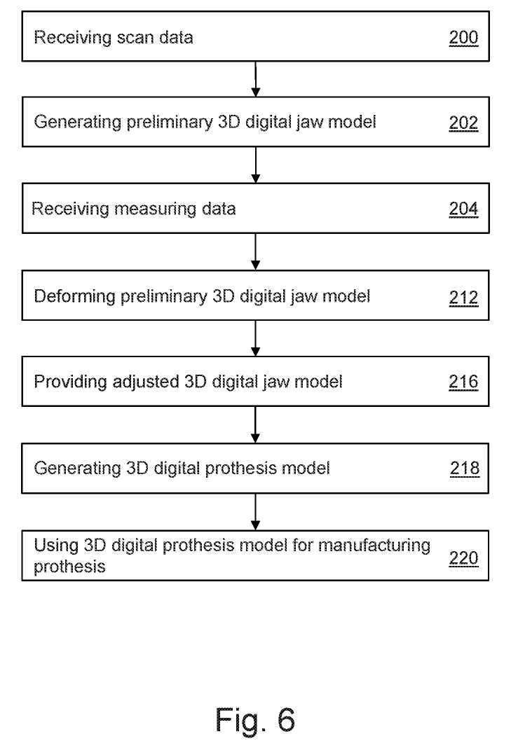

FIG. 6 shows an exemplary method for adjusting a preliminary three-dimensional digital jaw model of at least a part of a jaw of a patient. In block 200, scan data acquired using a scanning method are received. The scan data comprise a plurality of sets of imaging data of a plurality of patches of a surface of the at least part of the jaw with reference elements, e.g., implants, arranged within the at least part of the jaw. In block 202, a preliminary three-dimensional digital jaw model is generated. The generating comprises assembling the imaging data of the plurality of patches of the surface of the at least part of the jaw. For example, the preliminary three-dimensional digital jaw model is descriptive of preliminary positions of the reference elements. For example, reference elements represented by the preliminary three-dimensional digital jaw model have preliminary positions. In block 204, measuring data acquired using a measuring method are received. The measuring data is descriptive of relative positions of the reference elements arranged within the at least part of the jaw. In block 212, the preliminary three-dimensional digital jaw model is deformed. The deforming results in an adjusted three-dimensional digital jaw model with adjusted positions of the reference elements using the relative positions of the reference elements as references for the deforming. In block 216, the resulting adjusted three-dimensional digital jaw model is provided.

In block 218, a three-dimensional digital prosthesis model of an implant-based dental prosthesis is generated using the three-dimensional digital jaw model provided in block 216. The three-dimensional digital prosthesis model, e.g., comprises receptions for receiving fastening elements configured for fastening the implant-based dental prosthesis to implants, e.g., implants used as reference elements. The fastening elements may, e.g., comprise abutments and/or screws. For example, the implant-based dental prosthesis is an implant-based dental bridge and/or an implant-based denture. In block 220, the three-dimensional digital prosthesis model generated in block 218 is used as a template for controlling a manufacturing device to manufacture the implant-based dental prosthesis as a physical copy of the template.

Blocks 200, 202, 204, 212, and 216 of FIG. 6 may, e.g., correspond to blocks 200, 202, 204, 212, and 216 of any of FIGS. 2-5. Thus, the features disclosed in the context of the method of any of FIGS. 2-5 may also be comprised by and/or applied to the method of FIG. 6.

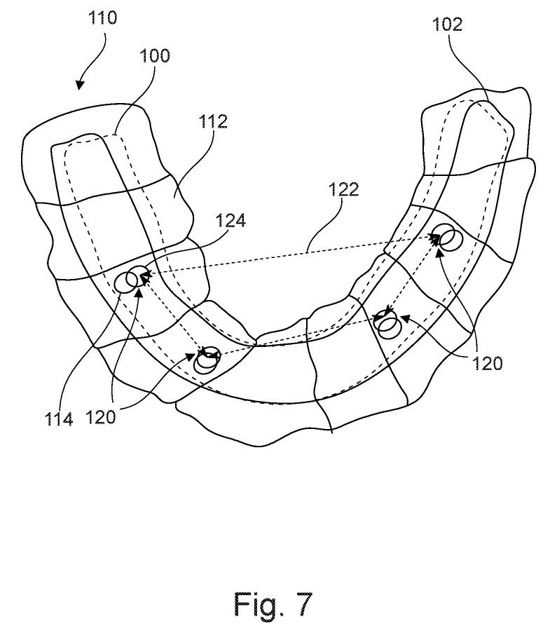

FIG. 7 shows an exemplary preliminary three-dimensional digital jaw model 102, which is deformed using relative positions 122 of implants as reference elements for providing an adjusted three-dimensional digital jaw model 100 with adjusted implant positions.

Scan data 110, which is acquired using a scanning method, comprises a plurality of sets of imaging data. Each of the sets of imaging data comprises a patch 112 of a surface of a jaw with implants arranged therein. The imaging data of the plurality of patches 112 of the surface of the jaw is assembled, such that a preliminary three-dimensional digital jaw model 102 is generated. The implants represented by the preliminary three-dimensional digital jaw model have preliminary implant positions 114. When the patches 112 of the surface of the jaw are being assembled, small errors may occur, e.g., regarding a relative positioning of adjacent patches 112. Such small errors may add up, such that deviations of the preliminary implant positions 114 from the actual positions of the implants in the jaw may occur. For example, the assembling of the patches 112 may comprise a registering of the imaging data of adjacent patches 112 with each other. Such an assembling using a registering may comprise errors to some degree, which may add up. In case a first patch is provided and a second patch is registered with respect to the first patch, a first error may occur. The first error may be descriptive of an error of position of a second surface section described by the second patch relative to an adjacent first surface section described by the first patch. When the second patch with the first error is used as a reference for registering a third patch with respect to this second patch, a second error may occur. However, since the position of the second patch already comprises an error, an error of position of a third surface section described by the third patch relative to the first surface section described by the first patch, i.e., a total error of position of the third surface section, may be a sum of the first and second error.

In order to correct such deviations, measuring data 120 may be used in addition to the scan data 110. The measuring data 120 is acquired using a measuring method. The measuring data 120 is at least descriptive of relative positions 122 of the implants as reference elements arranged within the at least part of the jaw. For example, the measuring data 120 may comprise measuring data of at least a section of the at least part of the jaw comprising all the implants. The relative positions 122 of the implants provided by the measuring data 120 may be used as references to adjust the preliminary implant positions 114.

The scan data 110 may provide a higher local accuracy for determining surface structures of the individual surface patches 112 compared to the measuring data 120, while the measuring data 120 may provide a higher global accuracy for determining the relative positions 122 of the implants compared to the scan data 110. For example, the scan data 110 may comprise optical scan data acquired using an optical scanning method. For example, the optical scan data of the scan data 110 may be acquired using an intraoral scan, a scan of an imprint of the at least part of the jaw, or a scan of a physical model of the at least part of the jaw, e.g., manufactured using an imprint. For example, the measuring data 120 may comprise optical measuring data acquired using an optical measuring method. For example, the optical measuring data of the measuring data 120 may be acquired using an optical measuring method, which is evaluated using a photogrammetric analysis, e.g., stereophotogrammetry. For example, the measuring data 120 may comprise tactile measuring data acquired using a tactile measuring method. For example, the measuring data 120 may comprise X-ray measuring data acquired using an X-ray based imaging method. For example, the X-ray based imaging method is an X-ray based three-dimensional imaging method, like CT or CBCT.

The preliminary three-dimensional digital jaw model 102 provided using the scan data 110 may be registered with the measuring data 120. The registering may comprise overlaying the measuring data 120 with the preliminary three-dimensional digital jaw model 102. For example, a relative position of the preliminary three-dimensional digital jaw model 102 relative to the measuring data 120 and/or vice versa may be adjusted until a mean deviation of the preliminary implant positions 114 of the implants in the preliminary three-dimensional digital jaw model 102 from the relative positions 122 of the implants in the measuring data 120 is minimized.

The preliminary three-dimensional digital jaw model 102 may then be deformed, e.g., until the adjusted implant positions 124 resulting from a relative shifting of the preliminary implant positions 114 due to the deforming coincide with the positions of the implants according to the relative positions 122. For example, the deforming may comprise using a Laplacian surface editing. The preliminary three-dimensional digital jaw model 102 may be provided by a three-dimensional digital mesh structure, which is adjusted to the reference positions described by the measuring data 120 using a mesh deformation. The deforming may result in an adjusted three-dimensional digital jaw model 100 with adjusted implant positions 124 coinciding with the reference markers provided by the relative positions 122 of the implants in the measuring data 120.

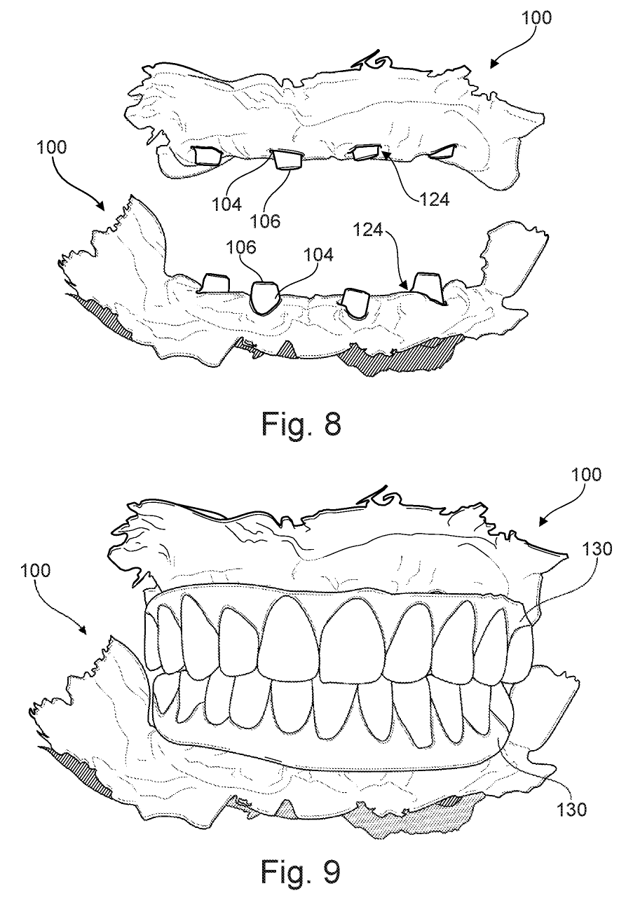

FIG. 8 shows exemplary adjusted three-dimensional digital jaw models 100 of a maxilla and a mandible. The three-dimensional digital jaw models 100 comprise reference elements in form of implants, i.e., three-dimensional digital implant models 104 arranged at positions, i.e., implant positions, provided by adjusted positions 124 of the reference elements, i.e., adjusted implant positions. In the example according to FIG. 8, the adjusted three-dimensional digital jaw models 100 further comprise three-dimensional digital abutment models 106 arranged within the implants within the respective jaw. The adjusted three-dimensional digital jaw models 100 may result from a deforming of preliminary three-dimensional digital jaw models, e.g., generated using scan data. For the deforming relative positions of reference elements, e.g., implants, provided by measuring data may be used as references, such that adjusted implant positions of the deformed preliminary three-dimensional digital jaw models coincide with the relative positions provided by the measuring data.

FIG. 9 shows exemplary three-dimensional digital prosthesis models 130 of implant-based dental prostheses. In FIG. 9, exemplary three-dimensional digital prosthesis models 130 of a maxilla and a mandible are shown. The three-dimensional digital prosthesis models 130 may, e.g., be generated using the adjusted three-dimensional digital jaw models 100 of the maxilla and the mandible. The adjusted three-dimensional digital jaw models 100 of FIG. 9 may, e.g., the same three-dimensional digital jaw models 100 as shown in FIG. 8. The three-dimensional digital prosthesis models 130 may, e.g., be generated such that they fit onto and between the adjusted three-dimensional digital jaw models 100. For example, the three-dimensional digital prosthesis models 130 may each comprise a support surface, which are configured as negatives of the surfaces of the adjusted three-dimensional digital jaw models 100, such that the three-dimensional digital prosthesis models 130 fit accurately onto the adjusted three-dimensional digital jaw models 100. For the generating of the three-dimensional digital prosthesis models 130, e.g., predefined three-dimensional digital prosthesis models and/or model parts, such as gingiva parts, teeth parts, and/or individual teeth, may be used. The predefined models and/or model parts may be adjusted to fit onto and/or between the adjusted three-dimensional digital jaw models 100. Such models and/or model parts may, e.g., be provided by predefined libraries and/or by scans of existing parts, in particular natural parts. Alternatively, the three-dimensional digital prosthesis models 130 may be generated from scratch.

The three-dimensional digital prosthesis models 130, e.g., comprise receptions (not shown) for receiving fastening elements configured for fastening the implant-based dental prostheses to the implants of the jaws. The fastening elements may, e.g., comprise abutments as shown in FIG. 8 and/or screws (not shown). The implant-based dental prostheses according to the three-dimensional digital prosthesis models 130 of FIG. 9 are implant-based dental bridges. More precisely, the implant-based dental prostheses are a maxillary and a mandibular implant-based denture. Implant-based dental bridges may, e.g., be bridges comprising full dental arches, i.e., implant-based dentures as shown in FIG. 9. Alternatively, implant-based dental bridges may, e.g., comprise only partial dental arches, i.e. part of dental arches.

The three-dimensional digital prosthesis models 130 may, e.g., be used as templates for controlling one or more manufacturing devices to manufacture implant-based dental prostheses as physical copies of the templates.

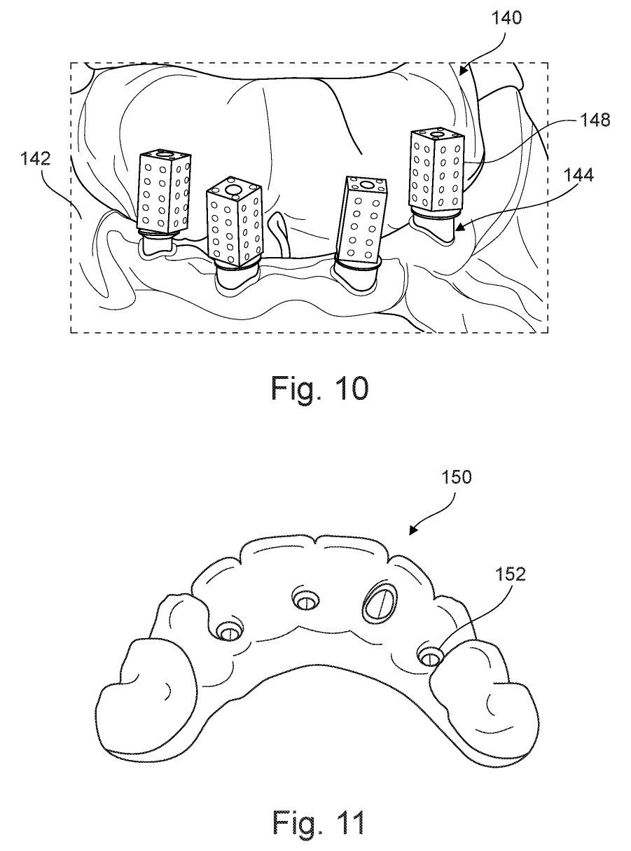

FIG. 10 shows an exemplary oral cavity 140 with a jaw bow 142. In the case of FIG. 10, a mandibular jaw bow is shown. In the jaw bow 142 implants 144 are arranged. On the implants 144 physical markers in form of dedicated physical fiducial markers 148 are arranged. These dedicated physical fiducial markers 148 may be used for facilitating a determining of relative positions of the implants within measuring data. The dedicated physical fiducial markers 148 may comprise predefined physical forms and surfaces, which may facilitate a determine of relative positions and/or orientations of the dedicated physical fiducial markers 148 and thus of the implants, on which the dedicated physical fiducial markers 148 are arranged. Alternatively, abutments, like the ones shown in FIG. 8, may be used for facilitating the determining of relative positions of the implants.

FIG. 11 shows an exemplary implant-based dental prosthesis 150. The implant-based dental prosthesis 150 is an implant-based dental bridge of a full maxillary dental arch, i.e., an implant-based maxillary denture. The implant-based dental prosthesis 150 comprises receptions 152 for receiving fastening elements, like abutments and/or screws, configured for fastening the implant-based dental prosthesis 150 to implants. The implant-based dental prosthesis 150 may, e.g., be manufactured using a three-dimensional digital prosthesis model as shown in FIG. 9. The positions of the receptions 152 within the implant-based dental prosthesis 150 may, e.g., be defined by adjusted implant positions of the implants of a three-dimensional digital jaw model 100, e.g., of a three-dimensional digital jaw model 100 as shown in FIG. 9. The three-dimensional digital prosthesis model of FIG. 9 may, e.g., be used as a template for controlling one or more manufacturing devices to manufacture the implant-based dental prosthesis of FIG. 11 as physical copies of the template.

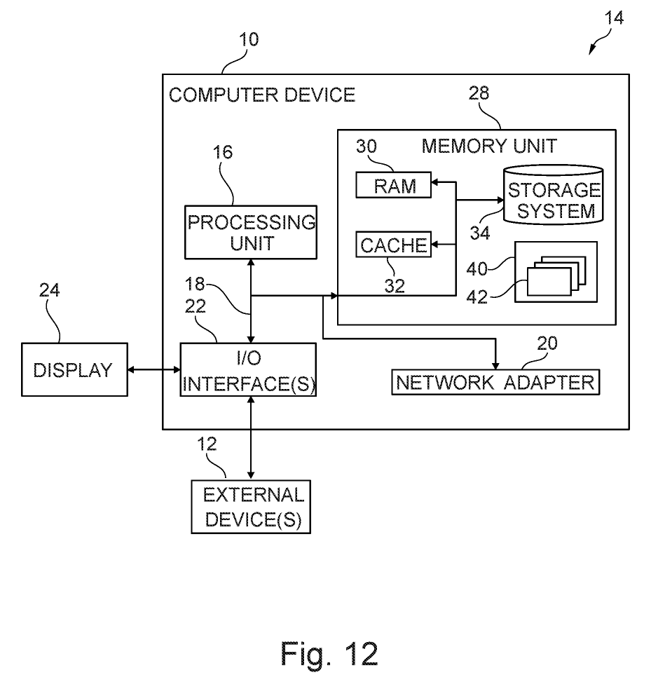

FIG. 12 shows a schematic diagram of an exemplary computer device 10 for adjusting a preliminary three-dimensional digital jaw model of at least a part of a jaw of a patient. The computer device 10 may be comprised by a computer system 14. The computer device 10 may be operational with numerous other general-purpose or special-purpose computing system environments and/or configurations. Computer device 10 may be described in the general context of computer device executable instructions, such as program modules comprising executable program instructions, being executable by the computer device 10. Generally, program modules may include routines, programs, objects, components, logic, data structures, and so on that perform particular tasks or implement particular abstract data types. Computer device 10 may be practiced in distributed computing environments where tasks are performed by remote processing devices that are linked through a communications network. In a distributed computing environment, program modules may be located in both local and remote computer device storage media including memory storage devices.

In FIG. 12, computer device 10 is, e.g., shown in the form of a general-purpose computer device. The components of computer device 10 may include, but are not limited to, one or more processors or processing units 16, a system memory or memory unit 28, and a bus 18 that couples various system components including memory unit 28 to processing unit 16. Bus 18 represents one or more of any of several types of bus structures, including a memory bus or memory controller, a peripheral bus, an accelerated graphics port, and a processing unit or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system 14 may comprise a variety of computer device readable storage media. Such media may be any available storage media accessible by computer device 10, and include both volatile and non-volatile storage media, removable and non-removable storage media.

A memory unit 28 may include computer device readable storage media in the form of volatile memory, such as random-access memory (RAM) 30 and/or cache memory 32. Computer device 10 may further include other removable/non-removable, volatile/non-volatile computer device storage media. For example, storage system 34 may be provided for reading from and writing to a non-removable, non-volatile magnetic media also referred to as a hard drive. For example, a magnetic disk drive for reading from and writing to a removable, non-volatile magnetic disk, e.g., a floppy disk, and an optical disk drive for reading from or writing to a removable, non-volatile optical disk such as a CD-ROM, DVD-ROM or other optical storage media may be provided. In such instances, each storage medium may be connected to bus 18 by one or more data media interfaces.

Memory unit 28 may, e.g., include scan data acquired using a scanning method. The scan data may, e.g., comprise a plurality of sets of imaging data of a plurality of patches of a surface of the at least part of the jaw with reference elements, e.g., implants, arranged within the at least part of the jaw. Memory unit 28 may, e.g., include measuring data acquired using a measuring method. The measuring data may be descriptive of relative positions of the reference elements arranged within the at least part of the jaw. The scan data may, e.g., provide a higher local accuracy for determining surface structures of the individual surface patches compared to the measuring data, while the measuring data may provide a higher global accuracy for determining the relative positions of the reference elements compared to the scan data. Memory unit 28 may, e.g., further include a preliminary three-dimensional digital jaw model generated using the scan data. The generating may, e.g., comprises an assembling of imaging data of the plurality of patches of the surface of the jaw comprised by the scan data. Memory unit 28 may, e.g., further include an adjusted three-dimensional digital jaw model with adjusted positions of the reference elements resulting from a deforming of the preliminary three-dimensional digital jaw model using the relative positions of the reference elements according to the measuring data as references for the deforming.

Memory unit 28 may, e.g., further include a three-dimensional digital prosthesis model of an implant-based dental prosthesis. The three-dimensional digital prosthesis model may comprise receptions for receiving fastening elements, like abutments and/or screws, configured for fastening the implant-based dental prosthesis to implants, e.g., implants used as reference elements. The three-dimensional digital prosthesis model may, e.g., be generated using the three-dimensional digital jaw model.

Program 40 may have a set of one or more program modules 42 and by way of example be stored in memory 28. The program modules 42 may comprise an operating system, one or more application programs, other program modules, and/or program data. Each of these program modules 42, i.e., the operating system, the one or more application programs, the other program modules, and/or the program data or some combination thereof, may include an implementation of a networking environment. One or more of the program modules 42 may be configured for adjusting a preliminary three-dimensional digital jaw model of at least a part of a jaw of a patient. The program modules 42 may, e.g., be configured for executing any of the methods of FIGS. 2-6.

Computer device 10 may further communicate with one or more external devices 12 of computer system 14 such as a keyboard, a pointing device, like a mouse, and a display 24 enabling a user to interact with computer device 10. Such communication can occur via input/output (I/O) interfaces 22 of computer device 10. Computer device 10 may further communicate with one or more networks such as a local area network (LAN), a general wide area network (WAN), and/or a public network, like the Internet, via network adapter 20. Network adapter 20 may communicate with other components of computer device 10 via bus 18. It should be understood that although not shown, other hardware and/or software components could be used in conjunction with computer device 10. For example, computer device 10 may provide one or more computing functions as a server, e.g., as a remote and/or cloud server.

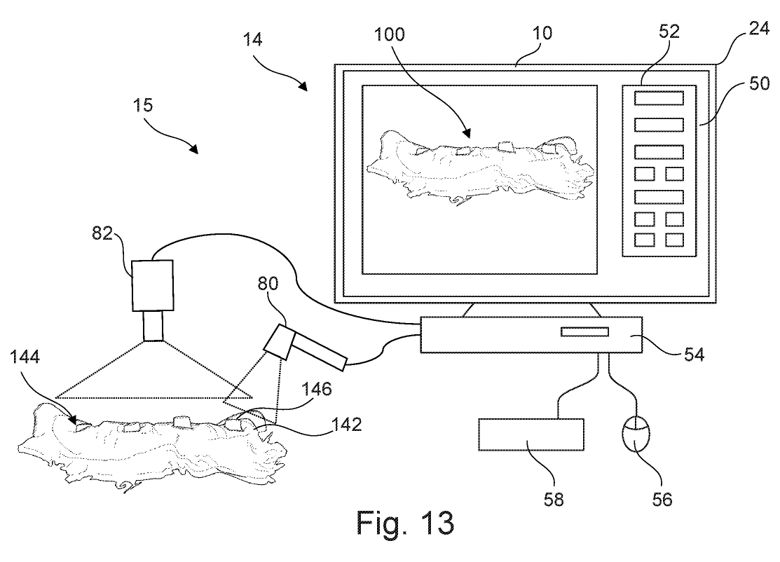

FIG. 13 shows an exemplary system 15 comprising a computer system 14 with a computer device 10. The computer device 10 is configured for adjusting a preliminary three-dimensional digital jaw model of at least a part of a jaw 142 of a patient. Thereby, an adjusted three-dimensional digital jaw model 100 of the at least part of the jaw 142 of the patient may be provided as a result. The jaw 142 may, e.g., comprise implants 144 and/or abutments 146 arranged therein. The computer device 10 may, e.g., be configured as shown in FIG. 12. The computer device 10 may comprise a hardware component 54 comprising one or more processing units as well as a memory storing machine-executable program instructions. Execution of the program instructions by the one or more processing units may cause the one or more processing units to control the computer device 10 to adjust a preliminary three-dimensional digital jaw model of at least a part of a jaw of a patient.

The computer system 14 may further comprise one or more input devices, like a keyboard 56 and a mouse 58, enabling a user to interact with the computer device 10. The one or more input devices may enable the user to control the adjusting of the preliminary three-dimensional digital jaw model of the at least part of the jaw of the patient. Furthermore, the computer system 14 may comprise one or more output devices, like a display 24 providing a graphical user interface 50 with control elements 52, e.g., GUI elements, enabling the user to control the adjusting of the three-dimensional digital jaw model of the at least part of the jaw of the patient. For example, scan data acquired using a scanning method (not shown) may be displayed on display 24. For example, a preliminary three-dimensional digital jaw model (not shown) generated using the scan data may be displayed on display 24 and deformed using relative positions of reference elements as references provided by measuring data acquired using a measuring method. For example, the preliminary three-dimensional digital jaw model and the measuring data (not shown) may be displayed on display 24 overlaid over each other. For example, the adjusted three-dimensional digital jaw model 100 of the at least part of the jaw 142 of the patient resulting from the adjusting of the preliminary three-dimensional digital jaw model may be displayed on display 24.

The system 15 may further comprise an exemplary scanning device 80 configured for acquiring scan data using a scanning method. The system 15 may further comprise an exemplary measuring device 82 configured for acquiring measuring data using a measuring method. The scan data may comprise a plurality of sets of imaging data of a plurality of patches of a surface of the at least part of the jaw with implants arranged within the at least part of the jaw. The measuring data may be descriptive of relative positions of the reference elements arranged within the at least part of the jaw. For example, the scan data acquired using the scanning device 80 provides a higher local accuracy for determining surface structures of the individual surface patches compared to the measuring data acquired using the measuring device 82, while the measuring data providing a higher global accuracy for determining the relative positions of the implants compared to the scan data.

For example, the scanning device 80 is an optical scanning device configured for acquiring scan data in form of optical scan data using an optical scanning method. For example, the optical scan data is acquired using an intraoral scan of the at least part of the jaw 142. Alternatively, the optical scan data may be acquired using a scan of an imprint of the at least part of the jaw 142 or of a physical model of the at least part of the jaw 142 manufactured, e.g., using an imprint.

For example, the scanning device 80 may be configured for scanning intraoral tissue within a patient’s oral cavity comprising the intraoral tissue. The intraoral tissue being scanned may, e.g., comprise hard and/or soft tissue. Hard tissue may, e.g., comprise teeth, while soft tissue may, e.g., comprise gingiva tissue. The intraoral tissue being scanned may, e.g., comprise one or more jaws of the patient, i.e., a mandible and/or a maxilla. A jaw being scanned may, e.g., be an edentulous jaw or a jaw comprising one or more teeth. Alternatively, the scanning device 80 may be configured for scanning an imprint of the intraoral tissue and/or a positive of the intraoral tissue generated using an imprint.

For example, the measuring device 82 is an optical measuring device configured for acquiring measuring data in form of optical measuring data using an optical measuring method. For example, the optical measuring data is evaluated using a photogrammetric analysis. For example, stereophotogrammetry is used. For example, the measuring data is acquired by measuring the at least part of the jaw 142. Alternatively, the measuring data may be acquired by measuring an imprint of the at least part of the jaw 142 or of a physical model of the at least part of the jaw 142 manufactured, e.g., using an imprint.

For example, the measuring device 82 is a tactile measuring device configured for acquiring tactile measuring data using a tactile measuring method. For example, the measuring data is acquired by measuring the at least part of the jaw 142. Alternatively, the measuring data may be acquired by measuring an imprint of the at least part of the jaw 142 or of a physical model of the at least part of the jaw 142 manufactured, e.g., using an imprint.

For example, the measuring device 82 is an X-ray imaging device configured for acquiring X-ray imaging data using an X-ray based imaging method. For example, the X-ray based imaging method is an X-ray based three-dimensional imaging method, e.g., CT or CBCT. For example, the measuring data is acquired by measuring the at least part of the jaw 142. Alternatively, the measuring data may be acquired by measuring an imprint of the at least part of the jaw 142 or of a physical model of the at least part of the jaw 142 manufactured, e.g., using an imprint.

For example, the measuring device 82 may be configured for measuring, e.g., scanning, intraoral tissue within a patient’s oral cavity comprising the intraoral tissue. The intraoral tissue being measuring may, e.g., comprise hard and/or soft tissue. Hard tissue may, e.g., comprise teeth, while soft tissue may, e.g., comprise gingiva tissue. The intraoral tissue being scanned may, e.g., comprise one or more jaws of the patient, i.e., a mandible and/or a maxilla. A jaw being measuring may, e.g., be an edentulous jaw or a jaw comprising one or more teeth. Alternatively, the measuring device 82 may be configured for measuring an imprint of the intraoral tissue and/or a positive of the intraoral tissue generated using an imprint.

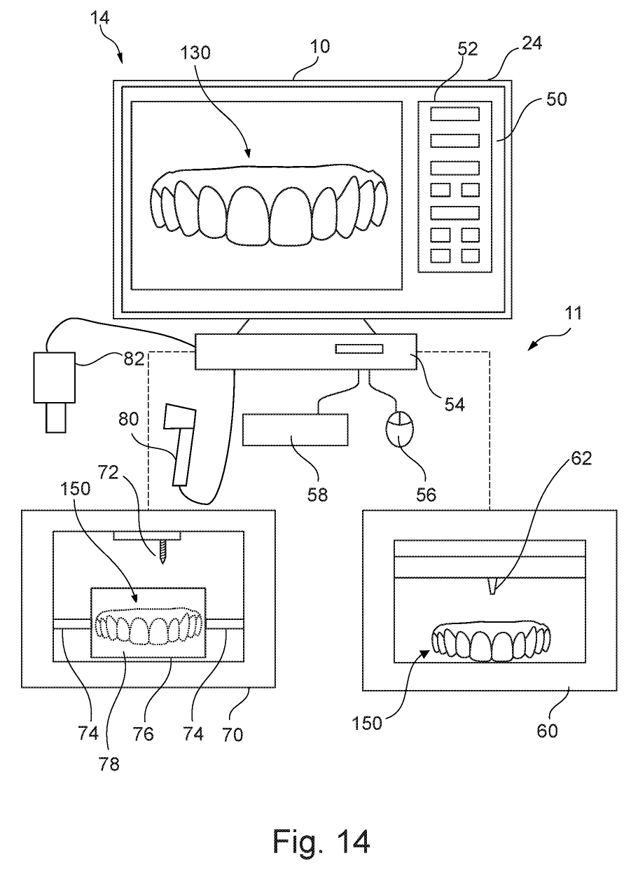

FIG. 14 shows an exemplary manufacturing system 11 for manufacturing an implant-based dental prosthesis 150 or at least one or more elements of the implant-based dental prosthesis 150. The implant-based dental prosthesis 150 may, e.g., be an implant-based dental bridge. The implant-based dental bridge may, e.g., be an implant-based denture. A three-dimensional digital prosthesis model 130 generated using a three-dimensional digital jaw model is provided. The three-dimensional digital jaw model may, e.g., be provided using any of the methods of FIGS. 2-6. The three-dimensional digital prosthesis model 130 may comprise receptions for receiving fastening elements, like abutments and/or screws, configured for fastening the implant-based dental prosthesis to implants. The three-dimensional digital prosthesis model 130 may be used as a template for controlling one or more manufacturing devices 70, 60 to manufacture the implant-based dental prosthesis 150 as a physical copy of the template.

The manufacturing system 11 may comprise one or more manufacturing devices 60, 70. The manufacturing system 11 may further comprise system 15 of FIG. 13 with the computer device 10 of FIGS. 12 and/or 13. The computer device 10 may further be configured to control the one or more manufacturing devices 60, 70. For example, the manufacturing system 11 may comprise a manufacturing device in form of a machining device 70 controlled by the computer device 10. The machining device 70 may be configured to processing a blank 76 using one or more machining tools 72. The blank 76 of raw material 78, may be provided using a holding device 74 and cut into a desired shape and size of the element to be manufactured, e.g., the implant-based dental prosthesis 150 as defined by the three-dimensional digital prosthesis model 130 or at least a part thereof. The machining device 70 is, e.g., a milling device with the machining tool 72 being a milling tool.

For example, the manufacturing system 11 may comprise a manufacturing device in form of a 3D printing device 60. The 3D printing device 60 may be controlled by the computer device 10 and configured to print an element to be manufactured, e.g., the implant-based dental prosthesis 150 as defined by the three-dimensional digital prosthesis model 130 or at least a part thereof. The 3D printing device 60 may comprise a printing element 62 configured to print the respective element, e.g., the implant-based dental prosthesis 150, layer by layer. The printing element 62 may, e.g., comprise a nozzle configured for distributing printing material.

In case the element to be manufactured using the 3D printing device 60 is made using metal, the 3D printing device 60 may, e.g., be configured for executing selective laser sintering or melting. Selective laser sintering uses a laser for sintering a powdered material, aiming the laser automatically at points in space defined by a three-dimensional digital model of the element to be printed. The laser energy may result in a local sintering or melting of the powdered material, binding the material together to create a solid structure. For example, the printing element 62 of the 3D printing device 60 may comprise a laser and/or a distributing device for distributing the powdered material.

For example, the three-dimensional digital prosthesis model 130 may be used as a positive to define a negative of the implant-based dental prosthesis 150 or of at least a part thereof in form of a negative three-dimensional digital dental prosthesis model or in form of a negative three-dimensional model of a part of the dental prosthesis, respectively. The negative three-dimensional digital dental prosthesis model or the negative three-dimensional model of the part of the dental prosthesis may be used to manufacture, e.g., using machining device 70 or 3D printing device 60, one or more casting matrices. The one or more casting matrices may, e.g., be configured for casting the implant-based dental prosthesis 150 or of at least a part thereof, by inserting casting material into the casting matrix and curing the inserted casting material.

For example, a combination of manufacturing devices, e.g., machining device 70 or 3D printing device 60, may be used for manufacturing the implant-based dental prosthesis 150. For example, a first manufacturing device, e.g., 3D printing device 60, may be used for manufacturing the implant-based dental prosthesis 150 without receptions for receiving fastening elements, like abutments and/or screws. A second manufacturing device, e.g., machining device 70, may be used for inserting the receptions into the manufacturing the implant-based dental prosthesis 150 provided by the first manufacturing device without receptions.

While the invention has been illustrated and described in detail in the drawings and foregoing description, such illustration and description are to be considered illustrative or exemplary and not restrictive; the invention is not limited to the disclosed examples.

Other variations to the disclosed embodiments can be understood and effected by those skilled in the art in practicing the claimed invention, from a study of the drawings, the disclosure, and the appended claims. In the claims, the word "comprising" does not exclude other elements or steps, and the indefinite article "a" or "an" does not exclude a plurality. The mere fact that certain measures are recited in mutually different dependent claims does not indicate that a combination of these measures cannot be used to advantage. Any reference signs in the claims should not be construed as limiting the scope.

A single processor or other unit may fulfill the functions of several items recited in the claims. A computer program may be stored/distributed on a suitable medium, such as an optical storage medium or a solid-state medium supplied together with or as part of other hardware, but may also be distributed in other forms, such as via the Internet or other wired or wireless telecommunication systems.

As will be appreciated by one skilled in the art, aspects of the present invention may be embodied as an apparatus, method, computer program or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer-readable medium(s) having computer executable code embodied thereon. A computer program comprises the computer executable code or “program instructions”.

Any combination of one or more computer-readable medium(s) may be utilized. The computer-readable medium may be a computer-readable signal medium or a computer-readable storage medium. A “computer-readable storage medium” as used herein encompasses any tangible storage medium which may store instructions which are executable by a processor of a computer device. The computer-readable storage medium may be referred to as a computer-readable non-transitory storage medium. The computer-readable storage medium may also be referred to as a tangible computer-readable medium. For example, a computer-readable storage medium may also be able to store data which is able to be accessed by the processor of the computer device. Examples of computer-readable storage media include, but are not limited to: a floppy disk, a magnetic hard disk drive, a solid-state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. A further example of an optical disk may be a Blu-ray disk. The term computer-readable-storage medium also refers to various types of recording media capable of being accessed by the computer device via a network or communication link. For example, a data may be retrieved over a modem, over the internet, or over a local area network. Computer executable code embodied on a computer-readable medium may be transmitted using any appropriate medium, including but not limited to wireless, wireline, optical fiber cable, RF, etc., or any suitable combination of the foregoing.

A computer-readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device.

“Computer memory” or “memory unit” is an example of a computer-readable storage medium. Computer memory is any memory which is directly accessible to a processing unit. “Computer storage” or “storage” is a further example of a computer-readable storage medium. Computer storage is any non-volatile computer-readable storage medium. For example, computer storage may also be computer memory or vice versa.

A “processor” or “processing unit” as used herein encompasses an electronic component which is able to execute a program or machine-executable instruction or computer executable code. References to the computer device comprising “a processor” should be interpreted as possibly containing more than one processor or processing core. The processor may for instance be a multi-core processor. A processor may also refer to a collection of processors within a single computer device or distributed amongst multiple computer devices. The term computer device should also be interpreted to possibly refer to a collection or network of computer devices each comprising a processor or processors. The computer executable code may be executed by multiple processors that may be within the same computer device, or which may even be distributed across multiple computer devices.

Computer executable code may comprise machine-executable instructions or a program which causes a processor to perform an aspect of the present invention. Computer executable code for carrying out operations for aspects of the present invention may be written in any combination of one or more programming languages, including an object-oriented programming language such as Java, Smalltalk, C++ or the like and conventional procedural programming languages, such as the “C” programming language or similar programming languages and compiled into machine-executable instructions. In some instances, the computer executable code may be in the form of a high-level language or in a pre-compiled form and be used in conjunction with an interpreter which generates the machine-executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Generally, the program instructions can be executed on one processor or on several processors. In the case of multiple processors, they can be distributed over several different entities like clients, servers etc. Each processor could execute a portion of the instructions intended for that entity. Thus, when referring to a system or process involving multiple entities, the computer program or program instructions are understood to be adapted to be executed by a processor associated or related to the respective entity.

A “user interface” as used herein is an interface which allows a user or operator to interact with a computer or computer device. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer to indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, one or more switches, one or more buttons, and accelerometer are all examples of user interface components which enable the receiving of information or data from an operator.

A GUI element is a data object some of which’s attributes specify the shape, layout and/or behavior of an area displayed on a graphical user interface, e.g., a screen. A GUI element can be a standard GUI element such as a button, a text box, a tab, an icon, a text field, a pane, a check-box item or item group or the like. A GUI element can likewise be an image, an alphanumeric character, or any combination thereof. At least some of the properties of the displayed GUI elements depend on the data value aggregated on the group of data object said GUI element represents.

Aspects of the present invention are described with reference to flowchart illustrations and/or block diagrams of methods, apparatus (systems) and computer program products. It will be understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer, other programmable data processing apparatus, or other devices to function in a particular manner, such that the instructions stored in the computer-readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

Although the invention may have been described in reference to specific examples, it should be understood that the invention is not limited to these examples only and that many variations of these examples may be readily envisioned by the skilled person after having read the present disclosure. The invention may thus further be described without limitation and by way of example only by the following embodiments. The following exemplary embodiments may contain preferred embodiments. Accordingly, the term “feature combination” as used therein may refer to such a “preferred embodiment”.

Feature combination 1. A method for adjusting a preliminary three-dimensional digital jaw model of at least a part of a jaw of a patient, the method comprising:
receiving the preliminary three-dimensional digital jaw model of the at least part of the jaw, the preliminary three-dimensional digital jaw model being descriptive of preliminary positions of reference elements of the jaw;
receiving measuring data of an additional measurement of relative positions of the reference elements of the jaw;
deforming the preliminary three-dimensional digital jaw model, the deforming resulting in an adjusted three-dimensional digital jaw model with adjusted positions of the reference elements using the measured relative positions of the reference elements described by the measuring data as references for the deforming.

Feature combination 2. The method of feature combination 1, the deforming of the preliminary three-dimensional digital jaw model being executed iteratively.

Feature combination 3. The method of any of the previous feature combinations, the method further comprising registering the preliminary three-dimensional digital jaw model with the measuring data, the registering comprising:
overlaying the preliminary three-dimensional digital jaw model and the measuring data;
adjusting a relative position of the preliminary three-dimensional digital jaw model and the measuring data until a mean deviation of the preliminary positions of reference elements described by the preliminary three-dimensional digital jaw model from the relative positions of the reference elements of the measuring data is minimized.

Feature combination 4. The method of feature combination 3, the adjusting of the relative position of the preliminary three-dimensional digital jaw model and the measuring data being executed iteratively.

Feature combination 5. The method of any of the previous feature combinations, the receiving of the preliminary three-dimensional digital jaw model comprising generating the preliminary three-dimensional digital jaw model, the generating of the preliminary three-dimensional digital jaw model comprising receiving scan data of the at least part of the jaw, the scan data being used for the generating of the preliminary three-dimensional digital jaw model.

Feature combination 6. The method of feature combination 5, the scan data comprising a plurality of sets of imaging data of a plurality of patches of a surface of the at least part of the jaw with the reference elements, the generating of the preliminary three-dimensional digital jaw model comprising assembling the imaging data of the plurality of patches of the surface of the at least part of the jaw.

Feature combination 7. The method of feature combination 6, the assembling comprising a registering of the imaging data of adjacent patches of the plurality of patches with each other.

Feature combination 8. The method of feature combination 7, the method further comprising using pattern recognition for the registering of adjacent patches, the pattern recognition comprising one of the following: a two-dimensional pattern recognition, a three-dimensional pattern recognition.

Feature combination 9. The method of any of feature combinations 5 to 8, the scan data providing a higher local accuracy for determining surface structures of the individual surface patches compared to the measuring data, while the measuring data providing a higher global accuracy for determining the relative positions of the reference elements compared to the scan data.

Feature combination 10. The method of any of feature combinations 5 to 9, the scan data comprising one of the following: optical scan data acquired using an optical scanning method, X-ray scan data acquired using an X-ray based imaging method.

Feature combination 11. The method of any of the previous feature combinations, the measuring data comprising measuring data of at least a section of the at least part of the jaw comprising all the reference elements.

Feature combination 12. The method of any of the previous feature combinations, the measuring data comprising optical measuring data acquired using an optical measuring method.

Feature combination 13. The method of feature combination 12, the optical measuring method comprising an evaluation using a photogrammetric analysis of the optical measuring data being acquired.

Feature combination 14. The method of any of the previous feature combinations, the measuring data comprising tactile measuring data acquired using a tactile measuring method.

Feature combination 15. The method of any of the previous feature combinations, the measuring data comprising X-ray measuring data acquired using an X-ray based imaging method.

Feature combination 16. The method of any of the previous feature combinations, the reference elements comprising implants arranged within the at least part of the jaw.

Feature combination 17. The method of feature combination 16, using physical markers arranged on the implants for determining the relative positions of the implants within the measuring data.

Feature combination 18. The method of feature combination 17, the physical markers comprising one of the following: abutments, dedicated physical fiducial markers with predefined physical forms and surfaces.

Feature combination 19. The method of any of the previous feature combinations, the deforming comprising using a Laplacian surface editing.

Feature combination 20. The method of any of the previous feature combinations, the method further comprising generating a three-dimensional digital prosthesis model of an implant-based dental prosthesis using the adjusted three-dimensional digital jaw model.

Feature combination 21. The method of feature combination 20, the three-dimensional digital prosthesis model comprising receptions for receiving fastening elements configured for fastening the implant-based dental prosthesis to implants.

Feature combination 22. The method of any of feature combinations 20 to 21, the implant-based dental prosthesis being an implant-based dental bridge.

Feature combination 23. The method of any of feature combinations 20 to 21, the method further comprising: using the three-dimensional digital prosthesis model as a template for controlling a manufacturing device to manufacture the implant-based dental prosthesis as a physical copy of the template.

Feature combination 24. A computer program for adjusting a preliminary three-dimensional digital jaw model of at least a part of a jaw of a patient, the computer program product comprising program instructions being executable by a processing unit of a computer device to cause the processing unit to control the computer device to:
receive the preliminary three-dimensional digital jaw model of the at least part of the jaw, the preliminary three-dimensional digital jaw model being descriptive of preliminary positions of reference elements of the jaw;
receive measuring data of an additional measurement of relative positions of the reference elements of the jaw;
deform the preliminary three-dimensional digital jaw model, the deforming resulting in an adjusted three-dimensional digital jaw model with adjusted positions of the reference elements using the measured relative positions of the reference elements described by the measuring data as references for the deforming.

Feature combination 25. A computer program product for adjusting a preliminary three-dimensional digital jaw model of at least a part of a jaw of a patient, the computer program product comprising a non-transitory computer-readable storage medium having program instructions embodied therewith, the program instructions being executable by a processing unit of a computer device to cause the processing unit to control the computer device to:
receive the preliminary three-dimensional digital jaw model of the at least part of the jaw, the preliminary three-dimensional digital jaw model being descriptive of preliminary positions of reference elements of the jaw;
receive measuring data of an additional measurement of relative positions of the reference elements of the jaw;
deform the preliminary three-dimensional digital jaw model, the deforming resulting in an adjusted three-dimensional digital jaw model with adjusted positions of the reference elements using the measured relative positions of the reference elements described by the measuring data as references for the deforming.

Feature combination 26. A computer device for adjusting a preliminary three-dimensional digital jaw model of at least a part of a jaw of a patient, the computer device comprising a processing unit and a memory unit storing program instructions executable by the processing unit, an execution of the program instructions by the processing unit causing the processing unit to control the computer device to:
receive the preliminary three-dimensional digital jaw model of the at least part of the jaw, the preliminary three-dimensional digital jaw model being descriptive of preliminary positions of reference elements of the jaw;
receive measuring data of an additional measurement of relative positions of the reference elements of the jaw;
deform the preliminary three-dimensional digital jaw model, the deforming resulting in an adjusted three-dimensional digital jaw model with adjusted positions of the reference elements using the measured relative positions of the reference elements described by the measuring data as references for the deforming.

### REFERENCE SIGNS LIST

10 computer device
11 manufacturing system
12 external device
14 computer system
15 system
16 processing unit
18 bus
20 network adapter
22 I/O interface
24 display
28 memory unit
30 RAM
32 cache
34 storage system
40 program
42 program module
50 user interface
52 control elements
54 hardware component
56 keyboard
58 mouse
60 3D printing device
62 printing element
70 machining device
72 machining tool
74 holding device
76 blank
78 raw material
80 scanning device
82 measuring device
100 adjusted 3D digital jaw model
102 preliminary 3D digital jaw model
104 3D digital implant model
106 3D digital abutment model
110 scan data
112 imaging data of surface patch
114 preliminary position
120 measuring data
122 relative positions
124 adjusted positions
130 3D digital prosthesis model
140 oral cavity
142 jaw bow
144 implant
146 abutment
148 dedicated physical fiducial marker
150 implant-based dental prosthesis
152 reception

1. A method for adjusting a preliminary three-dimensional digital jaw model of at least a part of a jaw of a patient, the method comprising:
receiving the preliminary three-dimensional digital jaw model of the at least part of the jaw, the preliminary three-dimensional digital jaw model being descriptive of preliminary positions of reference elements of the jaw;
receiving measuring data of an additional measurement of relative positions of the reference elements of the jaw;
deforming the preliminary three-dimensional digital jaw model, the deforming resulting in an adjusted three-dimensional digital jaw model with adjusted positions of the reference elements using the measured relative positions of the reference elements described by the measuring data as references for the deforming.

2. The method of claim 1, the deforming of the preliminary three-dimensional digital jaw model being executed iteratively.

3. The method of claim 1, the method further comprising registering the preliminary three-dimensional digital jaw model with the measuring data, the registering comprising:
overlaying the preliminary three-dimensional digital jaw model and the measuring data;
adjusting a relative position of the preliminary three-dimensional digital jaw model and the measuring data until a mean deviation of the preliminary positions of reference elements described by the preliminary three-dimensional digital jaw model from the relative positions of the reference elements of the measuring data is minimized.

4. The method of claim 3, the adjusting of the relative position of the preliminary three-dimensional digital jaw model and the measuring data being executed iteratively.

5. The method of claim 1, the receiving of the preliminary three-dimensional digital jaw model comprising generating the preliminary three-dimensional digital jaw model, the generating of the preliminary three-dimensional digital jaw model comprising receiving scan data of the at least part of the jaw, the scan data being used for the generating of the preliminary three-dimensional digital jaw model.

6. The method of claim 5, the scan data comprising a plurality of sets of imaging data of a plurality of patches of a surface of the at least part of the jaw with the reference elements, the generating of the preliminary three-dimensional digital jaw model comprising assembling the imaging data of the plurality of patches of the surface of the at least part of the jaw.

7. The method of claim 6, the assembling comprising a registering of the imaging data of adjacent patches of the plurality of patches with each other.

8. The method of claim 5, the scan data providing a higher local accuracy for determining surface structures of the individual surface patches compared to the measuring data, while the measuring data providing a higher global accuracy for determining the relative positions of the reference elements compared to the scan data.

9. The method of claim 5, the scan data comprising one of the following: optical scan data acquired using an optical scanning method, X-ray scan data acquired using an X-ray based imaging method.

10. The method of claim 1, the measuring data comprising optical measuring data acquired using an optical measuring method.

11. The method of claim 10, the optical measuring method comprising an evaluation using a photogrammetric analysis of the optical measuring data being acquired.

12. The method of claim 1, the measuring data comprising tactile measuring data acquired using a tactile measuring method.

13. The method of claim 1, the measuring data comprising X-ray measuring data acquired using an X-ray based imaging method.

14. The method of claim 1, the reference elements comprising implants arranged within the at least part of the jaw.

15. The method of claim 14, using physical markers arranged on the implants for determining the relative positions of the implants within the measuring data.

16. The method of claim 1, the deforming comprising using a Laplacian surface editing.

17. The method of claim 1, the method further comprising generating a three-dimensional digital prosthesis model of an implant-based dental prosthesis using the adjusted three-dimensional digital jaw model, the three-dimensional digital prosthesis model comprising receptions for receiving fastening elements configured for fastening the implant-based dental prosthesis to implants.

18. The method of claim 17, the method further comprising: using the three-dimensional digital prosthesis model as a template for controlling a manufacturing device to manufacture the implant-based dental prosthesis as a physical copy of the template.

19. A computer program product for adjusting a preliminary three-dimensional digital jaw model of at least a part of a jaw of a patient, the computer program product comprising a non-transitory computer-readable storage medium having program instructions embodied therewith, the program instructions being executable by a processing unit of a computer device to cause the processing unit to control the computer device to:
receive the preliminary three-dimensional digital jaw model of the at least part of the jaw, the preliminary three-dimensional digital jaw model being descriptive of preliminary positions of reference elements of the jaw;
receive measuring data of an additional measurement of relative positions of the reference elements of the jaw;
deform the preliminary three-dimensional digital jaw model, the deforming resulting in an adjusted three-dimensional digital jaw model with adjusted positions of the reference elements using the measured relative positions of the reference elements described by the measuring data as references for the deforming.

20. A computer device for adjusting a preliminary three-dimensional digital jaw model of at least a part of a jaw of a patient, the computer device comprising a processing unit and a memory unit storing program instructions executable by the processing unit, an execution of the program instructions by the processing unit causing the processing unit to control the computer device to:
receive the preliminary three-dimensional digital jaw model of the at least part of the jaw, the preliminary three-dimensional digital jaw model being descriptive of preliminary positions of reference elements of the jaw;
receive measuring data of an additional measurement of relative positions of the reference elements of the jaw;
deform the preliminary three-dimensional digital jaw model, the deforming resulting in an adjusted three-dimensional digital jaw model with adjusted positions of the reference elements using the measured relative positions of the reference elements described by the measuring data as references for the deforming.
